(19) United States
(12) Patent Application Publication
(10) Pub. No.: US 2023/0081369 A1
(43) Pub. Date: Mar. 16, 2023

(54) IFIT Polypeptides and Uses for Treating Tuberculosis Infection

(71) Applicant: Stellenbosch University, Stellenbosch (ZA)

(72) Inventors: Abhilasha Madhvi Mishra, Stellenbosch (ZA); Bienyameen Baker, Stellenbosch (ZA)

(73) Assignee: Stellenbosch University, Stellenbosch (ZA)

(21) Appl. No.: 17/767,734

(22) PCT Filed: Oct. 8, 2020
(86) PCT No.: PCT/IB2020/059466

§ 371 Date: Apr. 8, 2022

(30) Foreign Application Priority Data
Oct. 8, 2019 (ZA) 2019/06603

(51) Int. Cl. A61K 38/17; C12N 15/79; C07K 14/47; A61P 31/06

(52) U.S. Cl. CPC A61K 38/17; C12N 15/79; C07K 14/47; A61P 31/06

(57) ABSTRACT

The present invention relates to methods of increasing the cellular concentration of Interferon Induced Protein with Tetratricopeptide repeats (IFIT) polypeptides in a cell infected with a mycobacterium. The method includes the introduction of exogenous IFIT polypeptides or expression vectors encoding the exogenous IFIT polypeptides into the cell, wherein increasing the cellular concentration of the IFIT polypeptide reduces the number of viable mycobacteria in the cell. The invention also relates to methods of treatment and uses of IFIT proteins and uses of vectors encoding IFIT proteins.

### BACKGROUND OF THE INVENTION

Tuberculosis (TB) is a disease that is recalcitrant to effective clinical management. Its robust nature combined with outdated diagnostic methods have made treatment at the point of care increasingly difficult, especially in the ever-increasing prevalence of drug resistant strains.

Tuberculosis is the biggest poverty related disease and a major cause of infection-related mortality worldwide. The disease affects the vulnerable and tends to impact heavily on the poorest and most marginalised groups i.e. migrants, prisoners, homeless, smokers, drug & alcohol addicts and people with weak immune systems.

According to the World Health Organization (WHO) global TB report in 2018, tuberculosis is the number one infection killer globally. In 2018 an estimated 1.5 million people who were HIV-negative died of TB.

Nearly one third of the global population is believed to be infected with TB. Of those infected, an estimated 5-10% develop active TB during their lifetime, while the remaining 90% successfully contain the bacteria. Also, some of the close household contacts of TB patients remain uninfected and healthy, indicating that immune system is capable in controlling TB. Studying host immune response towards Mycobacterium tuberculosis (M. tb) can reveal the reasons behind the enigma of infection and active disease.

The host immune response has been studied in detail over the past years and has been found to vary for different strains of mycobacteria. Pathogenic mycobacteria are known to survive inside the host by arresting phagosome maturation. The pathogen survives inside the optimal environment of the phagosome and the mechanism responsible for its growth and survival inside the host is still unknown.

The causative agent of tuberculosis, Mycobacterium tuberculosis has the ability to survive inside the host's defence cells (macrophages) that are supposed to destroy it. The current drug regimen against TB still leads to the emergence of drug-resistant strains of Mycobacterium tuberculosis. The increasing incidence of multi-drug resistant (MDR) and extensively drug-resistant (XDR) strains suggest that new approaches to treatment are required. This has motivated research into the development of new anti-TB drugs. A detailed understanding of this response to Mycobacterium tuberculosis infection is necessary in order to elucidate host components and pathways that are manipulated by Mycobacterium tuberculosis to ensure its' survival. Host-directed therapeutics (HDTs) has the potential to improve tuberculosis therapy since resistance to host machinery is unlikely to develop.

Conventional TB treatment regimens consist of multiple drugs, taken for prolonged periods of time. These medications, targeted at the mycobacterium, have a number of negative side-effects, and the development of TB strains resistant to these drugs is a huge concern.

Interferon Induced proteins with Tetratricopeptide repeats (IFIT) and 2′-5′-oligoadenylate synthetase (OAS) proteins are a family of antiviral proteins have been shown to confer immunity against viral infection. OASs gene family constitute of OAS1, OAS2 and OAS3 are part of interferon induced genes and exhibit cellular functions including induction of apoptosis, immune cell receptor modulation and autophagy. IFIT proteins have been shown to be conserved in vertebrates, with homologues having been identified in several organisms. These proteins are generally produced during viral infection, Interferon (IFN) treatment, and/or during pathogen recognition by the immune system. The mechanism of action of these proteins has been extensively studied during the course of viral infection. IFIT1 binds to non-self RNA, particularly capped non-self RNA transcripts which lack methylation on the first proximal nucleotide. IFIT1 consequently inhibits the translation or replication of the non-self RNA in an organism.

IFITs are known to prevent viral replication by binding and controlling the function of viral proteins and RNAs, but their role against bacteria is not well understood. The human IFIT family include four members bunched on chromosome 10, namely IFIT1 (ISG56), IFIT2 (ISG54), IFIT3 (ISG60 or IFIT 4) and IFIT5 (ISG58) (Fensterl & Sen, 2011). The first discovered member of IFIT family was human IFIT1 (a 56-kDa protein synthesized against stimulation of IFN), its corresponding gene is ISG56, was the first human ISGs to be cloned (Kusari & Sen, 1987). IFITs are weakly induced in response to type III IFN (IFN-γ) and strongly in response to type I IFNs (IFN-α/β) and type III IFNs (IFN-λs) (Der, Zhou, Williams, & Silverman, 1998). The IFIT protein can distinguish between cellular and viral RNAs, and it binds to viral mRNA 5′ ends whose caps lack 2′-O-methylation of the first ribose (Kumar et al., 2014).

IFIT1, IFIT2 and IFIT3 forms a complex and bind to 5′-ppp end of vesicular stomatitis virus (VSV), on the other hand replication of VSV was restored if gene expressing IFITs (1, 2 and 3) were knocked down. The role of IFITs in the anti-mycobacterial response of macrophages and other immune cells has not been described previously. The invention relates to the novel induction of IFITs to kill mycobacteria in host immune cells to be used as a stand-alone anti-tuberculosis therapy or to be used in combination with current first or second line drugs.

### SUMMARY OF THE INVENTION

The present invention relates to methods for increasing the cellular concentration of an Interferon Induced Protein with Tetratricopeptide repeats (IFIT) polypeptide in a cell infected with a mycobacterium and to methods of treatment of mycobacterial infection with IFIT polypeptides or vectors encoding IFIT polypeptides. The invention also relates to uses of IFIT proteins and uses of vectors encoding IFIT proteins to stimulate an increase of intercellular IFIT protein concentrations.

In a first aspect of the invention there is provided for a method of increasing the cellular concentration of an Interferon Induced Protein with Tetratricopeptide repeats (IFIT) polypeptide in a cell infected with a mycobacterium, the method comprising introducing an exogenous IFIT polypeptide or an expression vector comprising a polynucleotide encoding the exogenous IFIT polypeptide into the cell, wherein the exogenous IFIT polypeptide is selected from at least one exogenous IFIT1, IFIT2 or IFIT3 polypeptide, and wherein increasing the cellular concentration of the IFIT polypeptide reduces the number of viable mycobacteria in the cell.

In a first embodiment of the method of the invention the exogenous IFIT polypeptides comprises the following amino acid sequences IFIT1 (SEQ ID NO:1), IFIT2 (SEQ ID NO:2) and IFIT3 (SEQ ID NO:3).

In a second embodiment of the method the polynucleotide encoding the exogenous IFIT polypeptides comprise the following nucleic acid sequences of IFIT1 (SEQ ID NO:4), IFIT2 (SEQ ID NO:5) and IFIT3 (SEQ ID NO:6). It will be appreciated by those of skill in the art that due to the degeneracy of the genetic code variations of SEQ ID NO:4, SEQ ID:5 and SEQ ID NO:6 will result in the production of the same amino acids listed in SEQ ID NO:1, SEQ ID NO:2 and SEQ ID NO:3. Further, it will also be appreciated that those of skill in the art may codon optimise a nucleic acid sequence for expression in a specific setting, such as a specific cell type.

In a third embodiment of the method of the invention there is provided for additionally introducing a pharmaceutical compound selected from known mycobacterium therapeutics in selected from the group consisting of arginine, amikacin, bedaquiline, capreomycin, ciprofloxacin, clarithromycin, clavulanic acid, clofazimine, co-amoxiclav, cycloserine, enviomycin, ethambutol, ethionamide, imipenem, interferon-γ, isoniazid, kanamycin, levofloxacin, linezolid, meropenem, metronidazole, moxifloxacin, para-aminosalicylic acid, prochlorperazine, prothionamide, pyrazinamide, rifabutin, rifampicin, streptomycin, thioacetazone, thioridazine, vitamin D, and viomycin to the cell.

A fourth embodiment of the method invention contemplates that the mycobacteriaum is preferably a mycobacterium selected from the group consisting of Mycobacterium smegmatis, Mycobacterium bovis and Mycobacterium tuberculosis (including clinical strains).

In a further embodiment of the method of the invention it is appreciated that the cell is an immune cell, such as a macrophage or any other phagocytic cells (professional or non-professional), that include human or animal monocyte-derived macrophages.

In a second aspect of the invention there is provided for a method of treating a mycobacterial infection in a subject, the method comprising administering either (i) a therapeutically effective amount of an exogenous Interferon Induced Protein with Tetratricopeptide repeats (IFIT) polypeptide to the subject, or a therapeutically effective amount of an expression vector comprising a polynucleotide encoding the exogenous IFIT polypeptide to the subject. It will be appreciated that increasing the cellular concentration of the IFIT polypeptide in a cell infected with a mycobacterium, leads to a reduction in the number of viable mycobacteria in the cell, thereby treating the mycobacterial infection. It will further be appreciated that the exogenous IFIT polypeptide is selected from at least one exogenous IFIT1, IFIT2 or IFIT3 polypeptide.

In a first embodiment of the method of treatment of the invention the exogenous IFIT polypeptides may comprise and amino acid sequence of IFIT1 (SEQ ID NO:1), IFIT2 (SEQ ID NO:2) and IFIT3 (SEQ ID NO:3) or combinations thereof.

In a second embodiment of the method of treatment the polynucleotide encoding the exogenous IFIT1, IFIT2 and IFIT3 polypeptides may comprise a nucleic acid sequence of IFIT1 (SEQ ID NO:4), IFIT2 (SEQ ID NO:5) and IFIT3 (SEQ ID NO:6) or combination thereof. It will be appreciated by those skilled in the art that other nucleic acid sequences may encode the polypeptides of SEQ ID NO:1, SEQ ID NO:2 and SEQ ID NO:3 due to the degeneracy of the genetic code.

In a third embodiment of the method of treatment of the invention there is provided for additionally administering a pharmaceutical compound selected from the group consisting of pharmaceutical compounds that are known for use in the treatment of infection with a mycobacterium. The pharmaceutical compounds may be selected from the group consisting of arginine, amikacin, bedaquiline, capreomycin, ciprofloxacin, clarithromycin, clavulanic acid, clofazimine, co-amoxiclav, cycloserine, enviomycin, ethambutol, ethionamide, imipenem, interferon-γ, isoniazid, kanamycin, levofloxacin, linezolid, meropenem, metronidazole, moxifloxacin, para-aminosalicylic acid, prochlorperazine, prothionamide, pyrazinamide, rifabutin, rifampicin, streptomycin, thioacetazone, thioridazine, vitamin D, and viomycin and are administered to the subject in combination with the IFIT polypeptide or expression vector. It will be appreciated that the pharmaceutical compound and the IFIT polypeptide or expression vector may be administered to the subject either separately, concurrently or sequentially.

A fourth embodiment of the method of treatment of the invention contemplates that the mycobacterium is preferably a mycobacterium selected from the group consisting of Mycobacterium smegmatis, Mycobacterium bovis and Mycobacterium tuberculosis (including clinical strains).

In a fifth embodiment of the method of treatment of the invention it will be appreciated that the cell may be an immune cell, such as a macrophage or any other phagocytic cells (professional or non-professional), that include human or animal monocyte-derived macrophages.

In yet a further embodiment of the method of treatment of the invention there is provided for the subject being selected from the group consisting of a reptile, bird or mammal. Those of skill in the art will appreciate that the aforementioned organisms are all susceptible to mycobacterial infection. Preferably, the subject is a human.

In a third aspect of the invention there is provided for an exogenous Interferon Induced Protein with Tetratricopeptide repeats (IFIT) polypeptide for use or an expression vector comprising a polynucleotide encoding the exogenous IFIT polypeptide for use in a method of treating a mycobacterial infection in a subject, the method comprising administering a therapeutically effective amount of either (i) the exogenous IFIT polypeptide; or (ii) the expression vector to the subject, thereby increasing the cellular concentration of the IFIT polypeptide in a cell infected with a mycobacterium, resulting in a reduction in the number of viable mycobacteria in the cell, thereby treating the mycobacterial infection. It will be appreciated that the exogenous IFIT polypeptide is selected from at least one exogenous IFIT1, IFIT2 or IFIT3 polypeptide.

In a first embodiment of use of the polypeptide or use of the vector the exogenous IFIT polypeptides may comprise and amino acid sequence of IFIT1 (SEQ ID NO:1), IFIT2 (SEQ ID NO:2) and IFIT3 (SEQ ID NO:3).

In a second embodiment of the use of the polypeptide or use of the vector the polynucleotide encoding the exogenous IFIT1, IFIT2 and IFIT3 polypeptides may comprise a nucleic acid sequence of IFIT1 (SEQ ID NO:4), IFIT2 (SEQ ID NO:5) and IFIT3 (SEQ ID NO:6). It will be appreciated by those of skill in the art that other nucleic acid sequences may encode the polypeptides of SEQ ID NO:1, SEQ ID NO:2 and SEQ ID NO:3 due to the degeneracy of the genetic code.

In a third embodiment of the use of the polypeptide or use of the vector of the invention there is provided for additionally administering a pharmaceutical compound selected from the group consisting of pharmaceutical compounds that are known for use in the treatment of infection with a mycobacterium. The pharmaceutical compounds may be selected from the group consisting of arginine, amikacin, bedaquiline, capreomycin, ciprofloxacin, clarithromycin, clavulanic acid, clofazimine, co-amoxiclav, cycloserine, enviomycin, ethambutol, ethionamide, imipenem, interferon-γ, isoniazid, kanamycin, levofloxacin, linezolid, meropenem, metronidazole, moxifloxacin, para-aminosalicylic acid, prochlorperazine, prothionamide, pyrazinamide, rifabutin, rifampicin, streptomycin, thioacetazone, thioridazine, vitamin D, and viomycin and are administered to the subject in combination with the IFIT polypeptide or expression vector. It will be appreciated that the pharmaceutical compound and the IFIT polypeptide or expression vector may be administered to the subject either separately, concurrently or sequentially.

A fourth embodiment of the use of the polypeptide or use of the vector of the invention contemplates that the mycobacterium is preferably a mycobacterium selected from the group consisting of Mycobacterium smegmatis, Mycobacterium bovis and Mycobacterium tuberculosis.

In a fifth embodiment of the use of the polypeptide or use of the vector it will be appreciated that the cell may be an immune cell, such as a macrophage, more preferably the cell is a human monocyte-derived macrophage.

In yet a further embodiment of the use of the polypeptide or use of the vector of the invention there is provided for the subject being selected from the group consisting of a reptile, bird or mammal. Those of skill in the art will appreciate that the aforementioned organisms are all susceptible to mycobacterial infection. Preferably, the subject is a human.

### BRIEF DESCRIPTION OF THE FIGURES

Non-limiting embodiments of the invention will now be described by way of example only and with reference to the following figures:

FIG. 1: Depiction of methodology adopted for vector based knock-up of IFITs. Gene of interest was ligated into the mammalian vector which was digested at it unique sites at both 3′ and 5′ ends. This recombinant plasmid was overexpressed in E. coli and then projected for transfection in THP-1 cells. Abbreviations: pc=vector construct; GOI=gene of interest; XhoI=Xanthomonas holcicola; NheI=Neisseria mucosa heidelbergensis; E. coli=Escherichia coli; CFUs=colony forming units; qRT-PCR=reverse transcription polymerase chain.

FIG. 2: Agarose gel electrophoresis (0.8%) for confirmation of intactness of plasmids IFIT1, IFIT2 and IFIT3 run in duplicate. Abbreviations: IFIT=interferon-induced protein with tetratricopeptide repeats.

FIG. 3: Flag tagged mammalian expression vector (pcDNA3.1) cloned with IFIT1 into NheI and XhoI restriction sites. Abbreviations: pc=vector construct; XhoI=Xanthomonas holcicola; NheI=Neisseria mucosa heidelbergensis; IFIT=interferon-induced protein with tetratricopeptide.

FIG. 4: Flag tagged mammalian expression vector (pcDNA3.1) cloned with IFIT2 into NheI and XhoI restriction sites. Abbreviations: pc=vector construct; XhoI=Xanthomonas holcicola; NheI=Neisseria mucosa heidelbergensis; IFIT=interferon-induced protein with tetratricopeptide.

FIG. 5: Flag tagged mammalian expression vector (pcDNA3.1) cloned with IFIT3 into NheI and XhoI restriction sites. Abbreviations: pc=vector construct; XhoI=Xanthomonas holcicola; NheI=Neisseria mucosa heidelbergensis; IFIT=interferon-induced protein with tetratricopeptide.

FIG. 6: Depiction of knock-down of infected THP-1 cells using siRNA pre-mix. siRNA premix was added to the mycobacterial infected cells seeded in 48-well plate. This was further projected for studying the intracellular survival of mycobacteria using colony forming units (CFUs). Knocking-down was confirmed through qRT-PCR and Western blot. Abbreviations: siRNA=small interfering ribonucleic acid; CO₂=carbon dioxide; ° C.=degree Celsius; CFUs=colony forming units; QRT-PCR=reverse transcription polymerase chain reaction.

FIG. 7: CFUs obtained with and without knock-up and knock-down of IFITs in human monocyte-derived macrophages (hMDMs) infected with Mycobacterium smegmatis; A=12 hours after IFIT knock-up; B=12 hours after IFIT knock-down; C=24 hours after IFIT knock-up; D=24 hours after IFIT knock-down; A significant decrease in CFUs was observed upon knocking-up with IFITs while a CFUs increased significantly upon knocking-down with IFITs. This was observed at both 12 and 24 hours post M. smegmatis infection. KU=knock-up; KD=knock-down.

FIG. 8: CFUs obtained with and without knock-up and knock-down of IFITs in hMDMs infected with Mycobacterium bovis BCG; A=12 hours after IFIT knock-up; B=12 hours after IFIT knock-down; C=96 hours after IFIT knock-up; D=96 hours after IFIT knock-down; A significant decrease in CFUs was observed upon knocking-up with IFITs while CFUs increased significantly upon knocking-down with IFITs. This was observed at both 12 and 96 hours post BCG infection. KU=knock-up; KD=knock-down.

FIG. 9: CFUs obtained with and without knock-up and knock-down of IFITs in hMDMs infected with Mycobacterium tuberculosis R179; A=12 hours after IFIT knock-up; B=12 hours after IFIT knock-down; C=96 hours after IFIT knock-up; D=96 hours after IFIT knock-down; A significant decrease in CFUs was observed upon knocking-up with IFITs while CFUs increased significantly upon knocking-down with IFITs. This was observed at both 12 and 96 hours post R179 infection. KU=knock-up; KD=knock-down.

FIG. 10: [i] A pictorial representation of colony counts in hMDMs infected with Mycobacterium smegmatis with multiplicity of infection (MOI) of 1 (at 12-hours post-infection) [ii-iv] and further overexpressed with IFIT1, IFIT2 and IFIT3 depicting a significant decrease in colony counts

FIG. 11: Results of cytotoxicity analysis of hMDMs at 12 hours post Mycobacterium smegmatis infection. All cells had a viability of <85% throughout, depicting no cytotoxic effect in cells upon knock-up or knock-down. UI=uninfected, M. smeg=Mycobacterium smegmatis, KU=knock-up, KD=knock-down.

FIG. 12: Results of cytotoxicity analysis of hMDMs at 12 and 96 hours post Mycobacterium bovis BCG infection. All cells had a viability of <85% throughout, depicting no cytotoxic effect in cells upon knock-up or knock-down. UI=uninfected, KU=knock-up, KD=knock-down.

FIG. 13: Results of cytotoxicity analysis of hMDMs at 12 and 96 hours post Mycobacterium tuberculosis R179 infection. All cells had a viability of <85% throughout, depicting no cytotoxic effect in cells upon knock-up or knock-down. UI=uninfected, KU=knock-up, KD=knock-down.

FIG. 14: Comparison of relative expression after knock-down and knock-up of IFIT1, IFIT2 and IFIT3 in hMDMs infected with Mycobacterium smegmatis; UI=uninfected, KU=knock-up, KD=knock-down, * indicates p<0.001.

FIG. 15: Comparison of relative expression after knock-down and knock-up of IFIT1, IFIT2 and IFIT3 in hMDMs infected with Mycobacterium bovis BCG; UI=uninfected, KU=knock-up, KD=knock-down, * indicates p<0.001.

FIG. 16: Comparison of relative expression after knock-down and knock-up of IFIT1, IFIT2 and IFIT3 in hMDMs infected with Mycobacterium tuberculosis R179; UI=uninfected, KU=knock-up, KD=knock-down, * indicates p<0.001.

FIG. 17: Western blot confirming knock-down and knock-up of IFIT1 in THP-1 cells infected with Mycobacterium smegmatis, Mycobacterium bovis BCG and Mycobacterium tuberculosis R179 at 12 hours post-infection (IFIT1-55 KDa, GAPDH=37 KDa); UI=uninfected, KU=knock-up, KD=knock-down, GAPDH=Glyceraldehyde 3-phosphate dehydrogenase. Housekeeping gene GAPDH is used here as a biological control as GAPDH is stably and constitutively expressed in many tissues and cells.

FIG. 18: Amino acid sequence of Interferon Induced Protein with Tetratricopeptide repeats—IFIT1 (SEQ ID NO:1).

FIG. 19: Amino acid sequence of Interferon Induced Protein with Tetratricopeptide repeats—IFIT2 (SEQ ID NO:2).

FIG. 20: Amino acid sequence of Interferon Induced Protein with Tetratricopeptide repeats—IFIT3 (SEQ ID NO:3).

FIG. 21: Nucleic acid sequence of Interferon Induced Protein with Tetratricopeptide repeats—IFIT1 (SEQ ID NO:4).

FIG. 22: Nucleic acid sequence of Interferon Induced Protein with Tetratricopeptide repeats—IFIT2 (SEQ ID NO:5).

FIG. 23: Nucleic acid sequence of Interferon Induced Protein with Tetratricopeptide repeats—IFIT3 (SEQ ID NO:6).

FIG. 24: Heatmap of 19 differentially expressed transcripts with lowest false-discovery-rate analyzed by Ampliseq, hMDMs infected with Mycobacterium smegmatis, Mycobacterium bovis BCG or Mycobacterium tuberculosis R179 were compared to uninfected hMDMs, where Mycobacterium smegmatis shows maximum host response. The level of expression is depicted with black as maximum and white as minimum expression. Dendogram indicates sample clustering.

FIG. 25: Cytokine levels (pg/ml) with and without knock-up (vector-based over-expression) and knock-down of IFITs hMDMs infected with Mycobacterium smegmatis, Mycobacterium bovis BCG and Mycobacterium tuberculosis R179. (*) depicts significant (p≤0.05) expression upon comparing uninfected with knock-up/down.

FIG. 26: Knock-up of IFITs and OASs family at individual and synergy level to study their effect on BCG. A significant reduction (p<0.001) was observed upon knock-up of IFIT1, IFIT2 and IFIT3 individually as compared to BCG infected THP-1 cells at 24 hours after knock-up.

FIG. 27: Knock-up of IFITs and OASs family at individual and synergy level to study their effect on BCG. A significant reduction (p<0.001) was observed upon knock-up of IFIT1, IFIT2 and IFIT3 individually as compared to BCG infected THP-1 cells at 96 hours after knock-up.

### SEQUENCE LISTING

The nucleic acid and amino acid sequences listed in the accompanying sequence listing are shown using standard letter abbreviations for nucleotide bases, and the standard three letter abbreviations for amino acids. It will be understood by those of skill in the art that only one strand of each nucleic acid sequence is shown, but that the complementary strand is included by any reference to the displayed strand. In the accompanying sequence listing:

SEQ ID NO:1—Amino acid sequence of Interferon Induced Protein with Tetratricopeptide repeats—IFIT isoform 1 (IFIT1).

SEQ ID NO:2—Amino acid sequence of Interferon Induced Protein with Tetratricopeptide repeats—IFIT isoform 2 (IFIT2).

SEQ ID NO:3—Amino acid sequence of Interferon Induced Protein with Tetratricopeptide repeats—IFIT isoform 3 (IFIT3).

SEQ ID NO:4—Nucleic acid sequence of IFIT1.

SEQ ID NO:5—Nucleic acid sequence of IFIT2.

SEQ ID NO:6—Nucleic acid sequence of IFIT3.

SEQ ID NO:7—Nucleic acid target sequence 1 of IFIT1.

SEQ ID NO:8—Nucleic acid target sequence 2 of IFIT1.

SEQ ID NO:9—Nucleic acid target sequence 1 of IFIT2.

SEQ ID NO:10—Nucleic acid target sequence 2 of IFIT2.

SEQ ID NO:11—Nucleic acid target sequence 1 of IFIT3.

SEQ ID NO:12—Nucleic acid target sequence 2 of IFIT3.

SEQ ID NO:13—Nucleic acid sequence of Hs_GAPDH Forward primer.

SEQ ID NO:14—Nucleic acid sequence of Hs_GAPDH Reverse primer.

SEQ ID NO:15—Nucleic acid sequence of Hs_UBC Forward primer.

SEQ ID NO:16—Nucleic acid sequence of Hs_UBC Reverse primer.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

By “mycobacteria”, “mycobacterial” or “Mycobacterium” is meant a genus of Actinobacteria belonging to the family Mycobacteriaceae.

Mycobacteria can colonize their hosts without the hosts showing any adverse signs. For example, billions of people around the world have asymptomatic infections of Mycobacterium tuberculosis.

Mycobacterial infections are generally quite difficult to treat. This is due to the fact that mycobacteria contain an almost impenetrable cell wall, which is neither Gram negative nor positive. In addition, they are naturally resistant to a number of antibiotics that disrupt cell-wall biosynthesis. Due to their unique cell wall, they can survive long exposure to acids, alkalis, detergents, oxidative bursts, lysis by complement, and many antibiotics. Most mycobacteria are susceptible to the antibiotics clarithromycin and rifamycin, but over the years antibiotic-resistant strains have emerged.

The terms “protein,” “peptide” or “polypeptide” refers to any chain of two or more amino acids, including naturally occurring or non-naturally occurring amino acids or amino acid analogues, irrespective of post-translational modification (e.g., glycosylation or phosphorylation).

Those skilled in the art will appreciate that polypeptides, peptides or peptide analogues can be synthesised using standard chemical techniques, for instance, by automated synthesis using solution or solid phase synthesis methodology. Automated peptide synthesisers are commercially available and use techniques known in the art. Polypeptides, peptides and peptide analogues can also be prepared from their corresponding nucleic acid molecules using recombinant DNA technology.

The terms “nucleic acid”, “nucleic acid molecule” or “polynucleotide” encompass both ribonucelotides (RNA) and deoxyribonucleotides (DNA), including cDNA, genomic DNA, and synthetic DNA. A nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides.

The term “complementary” refers to two nucleic acids molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms, or by manual alignment and visual inspection. Such sequences are then said to be “substantially identical.”

As used herein a “substantially identical” sequence is an amino acid or nucleotide sequence that differs from a reference sequence only by one or more conservative substitutions, or by one or more non-conservative substitutions, deletions, or insertions located at positions of the sequence that do not destroy or substantially reduce the antigenicity of one or more of the expressed polypeptides or of the polypeptides encoded by the nucleic acid molecules. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polypeptide or polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

The term “gene of interest,” refers to a nucleic acid sequence comprising a nucleotide sequence which includes a transcription unit, and which can be transcribed and translated into a protein. Using the methods and/or assay of the present invention the expression of a gene of interest may be interrupted or silenced as a result of perturbation of a chromosomal contact in the cell.

The term “vector” refers to a means by which polynucleotides or gene sequences can be introduced into a cell. There are various types of vectors known in the art including plasmids, viruses, bacteriophages and cosmids. Generally polynucleotides or gene sequences are introduced into a vector by means of a cassette. The term “cassette” refers to a polynucleotide or gene sequence that is expressed from a vector, for example, the polynucleotide or gene sequences encoding the IFIT1, IFIT2 and IFIT3 polypeptides described herein. A cassette generally comprises a gene sequence inserted into a vector, which in some embodiments, provides regulatory sequences for expressing the polynucleotide or gene sequences. In other embodiments, the vector provides the regulatory sequences for the expression of the IFIT1, IFIT2 or IFIT3 polypeptides. In further embodiments, the vector provides some regulatory sequences and the nucleotide or gene sequence provides other regulatory sequences. “Regulatory sequences” include but are not limited to promoters, transcription termination sequences, enhancers, splice acceptors, donor sequences, introns, ribosome binding sequences, poly(A) addition sequences, and/or origins of replication.

The term “transcription” refers to the process of producing RNA from a DNA template. “In vitro transcription” refers to the process of transcription of a DNA sequence into RNA molecules using a laboratory medium which contains an RNA polymerase and RNA precursors and “intracellular transcription” refers to the transcription of a DNA sequence into RNA molecules, within a living cell. Further, “in vivo transcription” refers to the process of transcription of a DNA sequence into RNA molecules, within a living organism, such as a human subject.

The IFIT proteins and vectors encoding the IFIT proteins described herein may be used to treat mycobacterial infection or conditions associated with mycobacterial infection in a cell or in a subject. By “condition associated with mycobacterial infection” is meant any condition, disease or disorder that has been correlated with the presence of an existing mycobacterial infection.

As used herein the term “subject” includes animals, preferably the animal is a bird, reptile or mammal. Most preferably, the mammal is a human.

The IFIT proteins or vectors encoding the IFIT proteins described herein can be provided either alone or in combination with other compounds, in the presence of an adjuvant, or any carrier, such as a pharmaceutically acceptable carrier and in a form suitable for administration to a subject.

The term “pharmaceutically acceptable” refers to properties and/or substances which are acceptable for administration to a subject from a pharmacological or toxicological point of view. Further “pharmaceutically acceptable” refers to factors such as formulation, stability, patient acceptance and bioavailability which will be known to a manufacturing pharmaceutical chemist from a physical and/or chemical point of view.

The “suitable forms” of IFIT proteins and vectors encoding the IFIT proteins may be combined with “pharmaceutically acceptable carriers” and other elements known in the art in order to ensure efficient delivery of the IFIT proteins and vectors encoding the IFIT proteins to a subject or a cell.

By “pharmaceutically acceptable carrier” is meant a solid or liquid filler, diluent or encapsulating substance which may be safely used for the administration of pharmaceutically acceptable IFIT proteins and pharmaceutically acceptable vectors encoding the IFIT proteins to a subject or a cell.

As used herein a “pharmaceutically acceptable carrier” or “excipient” includes any and all antibacterial and antifungal agents, coatings, dispersion media, solvents, isotonic and absorption delaying agents, and the like that are physiologically compatible. A “pharmaceutically acceptable carrier” may include a solid or liquid filler, diluent or encapsulating substance which may be safely used for the administration of the composition to a subject or a cell. Suitable formulations or compositions to administer the IFIT proteins and vectors encoding the IFIT proteins to subjects who are to be prophylactically treated for a mycobacterial infection, who are suffering from a mycobacterial infection or subjects who are presymptomatic for a condition associated with mycobacterial infection fall within the scope of the invention. Any appropriate route of administration may be employed, such as, parenteral, intravenous, subcutaneous, intramuscular, intracranial, intraorbital, ophthalmic, intraventricular, intracapsular, intraspinal, intrathecal, intracistemal, intraperitoneal, intranasal, aerosol, topical, or oral administration.

Pharmaceutically acceptable carriers may include sterile aqueous solutions, dispersions and sterile powders for the preparation of sterile solutions. The use of media and agents for the preparation of pharmaceutically active substances is well known in the art. Where any conventional media or agent is incompatible with the active compound, use thereof in the pharmaceutical compositions of the invention is not contemplated. Supplementary active compounds can also be incorporated into the compositions.

An “effective amount” of the IFIT proteins and vectors encoding the IFIT proteins according to the invention includes a therapeutically effective amount, or a prophylactically effective amount. A “therapeutically effective amount” refers to an amount effective, at dosages and for periods of time necessary, to achieve the desired therapeutic result, such as treatment of a mycobacterial infection or a condition associated with such infection. The outcome of the treatment may for example be measured by a decrease in mycobacterial cell counts, inhibition of bacterial gene expression and replication, delay in development of a pathology associated with the mycobacterial infection, stimulation of the immune system, or any other method of determining a therapeutic benefit. A therapeutically effective amount of a compound may vary according to factors such as the disease state, age, sex, and weight of the subject, and the ability of a treatment to elicit a desired response in the individual. Dosage regimens may be adjusted to provide the optimum therapeutic response. A therapeutically effective amount is also one in which any toxic or detrimental effects of the treatment are outweighed by the therapeutically beneficial effects.

In the context of treating a condition the term “effective amount” refers to the administration of an amount of the active ingredients to an individual in need of treatment, either a single dose or several doses of the active ingredients may be administered to a subject.

Although some indications have been given herein as to suitable dosages of the IFIT proteins and vectors encoding the IFIT proteins of the invention, the exact dosage and frequency of administration of the effective amount will be dependent on several factors. These factors include the individual components used, the formulation of the extract or pharmaceutical composition containing the extract, the condition being treated, the severity of the condition, the age, body weight, health and general physical condition of the subject being treated, the nature and severity of the disorder to be treated or prevented, the route of administration, other medication that the subject may be taking, and other factors as are known to those skilled in the art. It is expected that the effective amount will fall within a relatively broad range that can be determined through routine trials.

Dosage values may vary with the severity of the condition to be alleviated. For any particular subject, specific dosage regimens may be adjusted over time according to the individual need and the judgment of the person administering or supervising the administration of the agents described herein. Dosage ranges set forth herein are exemplary only and do not limit the dosage ranges that may be selected. Dosage regimens may be adjusted to provide the optimum therapeutic response. For example, a single dose may be administered, or multiple doses may be administered over time. It may be advantageous to formulate the compositions in dosage unit forms for ease of administration and uniformity of dosage.

Toxicity and therapeutic efficacy of compositions of the invention may be determined by standard pharmaceutical procedures in cell culture or using experimental animals, such as by determining the LD₅₀ and the ED₅₀. Data obtained from the cell cultures and/or animal studies may be used to formulate a dosage range for use in a subject. The dosage of any composition of the invention lies preferably within a range of circulating concentrations that include the ED₅₀ but which has little or no toxicity. The dosage may vary within this range depending upon the dosage form employed and the route of administration utilised. For compositions of the present invention, the therapeutically effective dose may be estimated initially from cell culture assays.

The following example is offered by way of illustration and not by way of limitation.

### Example 1

Culture of THP-1 Cells for Knock-Up/Down Experiments

Human macrophage-like cells, THP-1 (ATCC-88081201), were cultured in RPMI-1640 supplemented with 10% heat-inactivated fetal calf serum (Biochrome, Germany). The cells were incubated at 37° C. in a 5% CO₂ incubator. THP-1 cells were treated with a final concentration of 100 nM Phorbol 12-Myristate 13-Acetate (PMA; Sigma Aldrich, USA) for 48 hours. Cells were transferred to CO₂ incubator in a BSL3 laboratory and proceeded with infection. For infection experiments, human macrophage cells were seeded in 12-well plates with 0.7×10⁶ cells per well.

Preparation of detergent-free mycobacteria for infection

The three different species of mycobacteria (Mycobacterium smegmatis, Mycobacterium bovis BCG and Mycobacterium tuberculosis R179 clinical isolate) were cultured separately in T25 flasks with 10 ml volume in each flask (to have an appropriate air space). These were incubated in a 37° C. incubator for 2-3 weeks. The subcultures were grown up to an optical density of 0.4 as maximum. Agitation was required for Mycobacterium smegmatis but not for Mycobacterium bovis BCG and Mycobacterium tuberculosis R179. The cultures were finally stocked at −80° C. for future use. Stock cultures of mycobacteria were brought out of −80° C. and thawed. Clumps in the thawed vials were disrupted by pipetting 10 times with 1 ml tip. This was then syringed 10 times (20 passes) through a G25 needle. This was then followed by allowing the major clumps to settle down for their respective settling time. The settling time was different for each strain (Mycobacterium smegmatis=30 seconds, Mycobacterium bovis BCG=1 minute, Mycobacterium tuberculosis R179=1 minute). The top 750 μl was collected and added to 4.25 ml of RPMI 1640 media. This 5 ml bacterial suspension was then immediately filtered through 5.0 μm pore size filter (Merck Millipore, Germany) to which 10% Human Serum was added. The required volume (according to the titration and MOI calculation) was then added to THP-1 cells in complete medium (RPMI1640+10% human serum). Mycobacterium tuberculosis stock titration was also done by this procedure (no human serum was added in this case), where an average CFU was obtained by processing 3 stock vials.

TABLE 1
Bacterial strains used in the study

| Strains/Plasmids | Description | Source/Reference |
|---|---|---|
| Mycobacterium | Mycobacterium | Laboratory collection |
| smegmatis | smegmatis | (Harper, Hayward, |
| | MC155 | Kidd, Wiid, & |
| | | van Helden, 2010) |
| Mycobacterium | Mycobacterium bovis | Laboratory collection |
| bovis BCG | BCG strain Pasteur | (Viljoen, Kirsten, Baker, |
| | 1743P2 | Helden, & Wiid, 2013) |
| Mycobacterium | Beijing genotype | Clinical isolate |
| tuberculosis | strain R220 | (Johnson et al., 2006) |
| R179 | | |
| E. coli DH5α | ATCC53868 | Laboratory collection |

Infection with Mycobacteria

Pathogenic (Mycobacterium tuberculosis R179), facultative-pathogenic (Mycobacterium bovis BCG) and non-pathogenic (Mycobacterium smegmatis) species of mycobacteria were used for infection. Mycobacteria were cultured in 7H9 (added 10% OADC and 0.5% glycerol) without Tween 80. The inventors avoid the use of Tween, as Tween is known to affect macrophage uptake and immune response to Mycobacterium tuberculosis. THP-1 cells were infected with each mycobacterial strain at MOI=1 and permitted for four hours of uptake. The cells were washed three times with phosphate buffered saline (PBS) to remove any extracellular mycobacteria.

Two separate in vitro infection experiments were setup, in the first experiment, cells were infected with respective mycobacterial species and then washed after 4 hours of infection. Cells were then incubated for another 8 hours in 5% CO₂ incubator at 37° C. Similarly, for another set of experiments, infected cells were washed after four hours of infection and then incubated for another 92 hours in 5% CO₂ incubator at 37° C.

Uninfected THP-1 cells served as control/uninfected samples. Downstream processing of cells was then carried out by knocking-up/down of IFIT1, IFIT2 and IFIT3 genes. Cells were then processed for CFU analysis (at 12 and 96 hours), RNA extraction at 12- and 96- hours and processed for Western Blot by collecting cell lysate upon treating with RIPA buffer containing Protease inhibitor at 12 hours and 96 hours post-infection. Cell supernatant was collected at 12 hours and 96 hours post-infection for Luminex assay.

Knock-Up of IFITs (IFIT1, IFIT2 and IFIT3)

Plasmids

pcDNA3.1 3×Flag IFIT1 (Addgene plasmid #53554, RRID:Addgene_53554), pcDNA3.1 3×Flag IFIT2 (Addgene plasmid #53555, RRID:Addgene_53555) and pcDNA3.1 3×Flag IFIT3 (Addgene plasmid #53553, RRID:Addgene_53553) plasmids were a gift from Kathleen Collins and were prepared as set out in Katibah et al (2013). Each of the respective plasmids contained the IFIT1, IFIT2 and IFIT3 genes.

Overexpression in E. coli

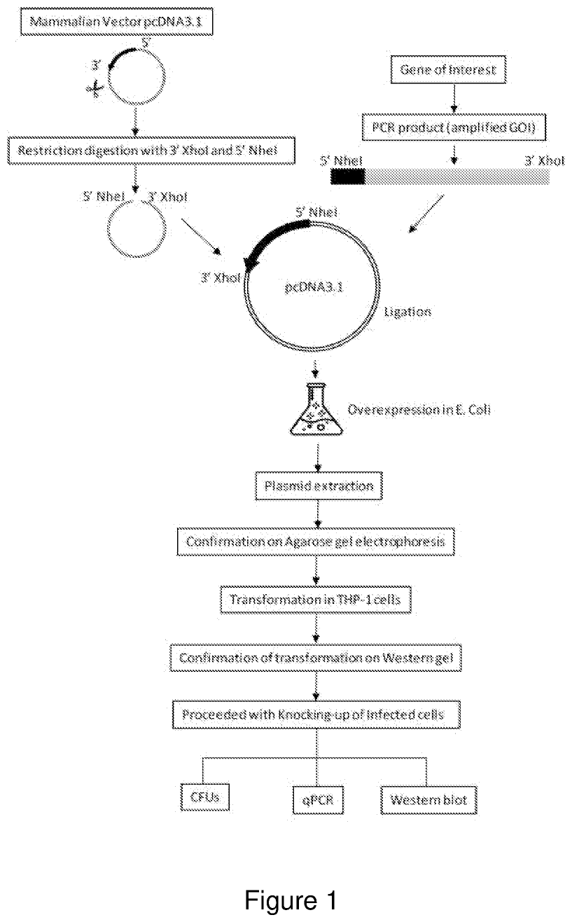

The bacterial stock containing cloned plasmid was streaked on LB agar plate with 100 μg/ml of ampicillin antibiotic as the plasmid designed was resistant to Ampicillin antibiotic. The agar plate was incubated overnight at 37° C. in a 5% CO₂ incubator. A single colony was picked up and inoculated overnight in LB broth at 37° C. in a 5% CO₂ shaking incubator. The final product, which was designed with Ampicillin resistance, was sequenced in order to verify the correct plasmid. FIG. 1 shows the detailed flow of vector based knock-up of IFITs.

Plasmid Extraction

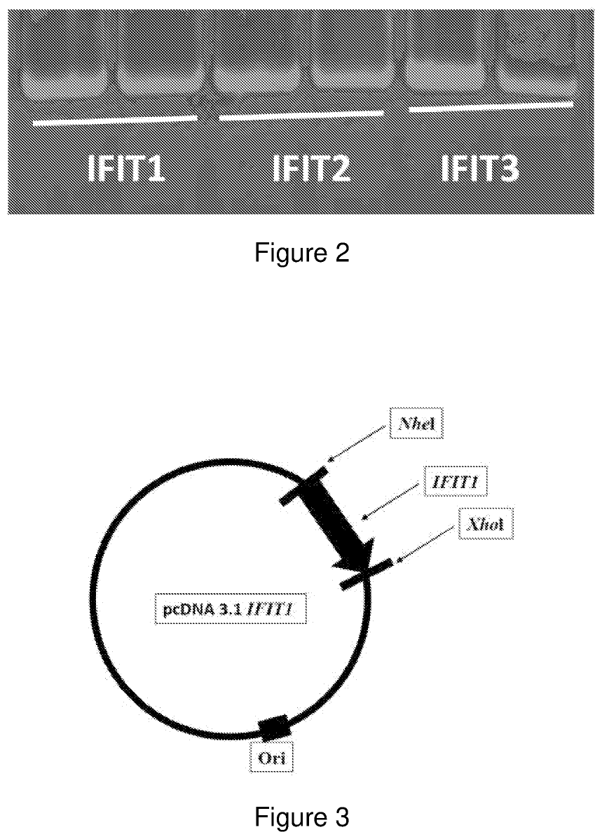

Plasmid extraction was carried out for the high-copy plasmid using the QIAGEN Plasmid Mini Kit. The starter LB Broth culture was processed by spinning at 6000×g for 15 minutes at 4° C. in order to harvest the bacterial cells. The bacterial cells were re-suspended in 0.3 ml of Buffer P1. 0.3 ml of Buffer P2 was mixed thoroughly by vigorously inverting the sealed tube 4-6 times and incubating at room temperature for 5 minutes. 0.3 ml of ice-cold buffer P3 was added immediately and mixed thoroughly in order to neutralize the solution completely. This mixture was incubated on ice for 5 minutes. The mixture was centrifuged at a maximum speed in a micro centrifuge for 10 minutes. This helps to separate the supernatant which contains plasmid DNA. This was added to a QIAGEN column to bind the DNA to the column. This column was washed with 2 ml of Buffer QC. Plasmid DNA was finally eluted with 0.5 ml of double distilled water. The purity and DNA intactness was analysed using 0.8% agarose gel electrophoresis. FIG. 2 depicts the Agarose gel with Plasmids IFIT1, IFIT2 and IFIT3.

Transfection

Optimization of Transfection in THP-1 cells was performed using different transfection reagents including Lipofectamine 2000 (ThermoFisher Scientific, Cat. No. 11668019), Lipofectamine 3000 (ThermoFisher Scientific, Cat. No. L3000015), Mission siRNA Transfection Reagent (Sigma-Aldrich, Cat No: S1452). A successful plasmid transfection in THP-1 cells was achieved by Mission siRNA transfection reagent which was confirmed by Western Blot. Transfection was performed in a 48 well plate (Greiner Bio One, cat. No. 677180) with mycobacteria infected 0.05×10⁶ cells/well in a 300 μl volume of complete media (RPMI+10% FBS).

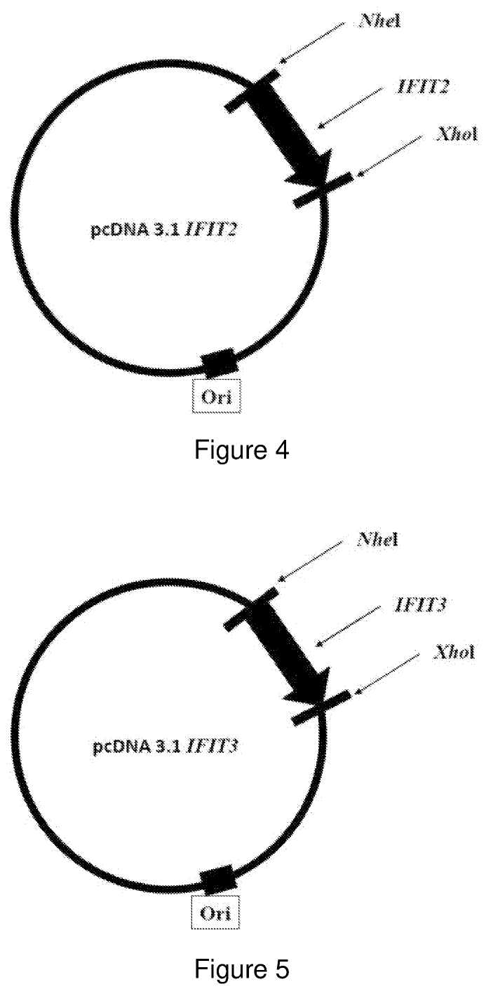

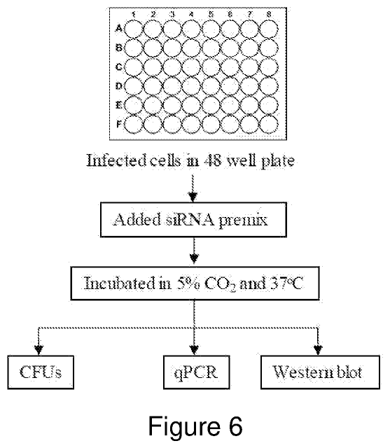

For 1 well of a 48 well plate, 2 μl of Transfection reagent was added to 125 ng of plasmid DNA (titrated for transfection) and finally mixed with 100 μl of DMEM. This mixture was vortexed and incubated for 15 minutes at room temperature. 100 μl of the final mixture was added to each well and the plate was swirled slowly for mixing. Vector only (pcDNA3.1) was used as a control in this experiment. The cells were then incubated at 37° C. in a 5% CO₂ incubator for the respective time points. Detailed representation of plasmid for IFIT1, IFIT2 and IFIT3 are provided in FIG. 3, FIG. 4 and FIG. 5.

Knock-Down of IFITs (IFIT1, IFIT2 and IFIT3)

THP-1 cells were seeded at 5×10⁴ cells per well of a 48 well plate in 0.3 ml of RPMI supplemented with 10% FBS. 100 nM of a final concentration of PMA was added and mixed well. Cells were incubated for 18-20 hours at 37° C. in a 5% CO₂ incubator. After incubation, cells were infected with respective mycobacterial strains at pre-specified MOIs (MOI=1).

After 4 hours of bacterial uptake, cells were washed thoroughly with PBS and fresh complete media was added to each well (300 μl per well of a 48 well plate). 6.25 μl Flexi Tube siRNA Premix was added drop-wise to the cells which made a final siRNA concentration of 25 nM in each well (Table 2 provides the detailed list of siRNA premix used). For negative control, a scrambled sequence was used as a negative siRNA Premix (Cat. No. S103650325). Culture plates were swirled slowly to mix the premix in complete media. Cells were incubated with the transfection complexes under their optimal growth conditions (37° C. in a 5% CO₂ incubator). Gene silencing was measured at 24 hours and 96 hours post transfection through intra cellular CFUs. Silencing was performed using Qiagen FlexiTube siRNA Premix. In vitro gene silencing for each gene was performed targeting two different silencing sites. The latter results including CFUs and mRNA expression levels was taken as an average of the values generated by silencing two different target sequences.

TABLE 2
Detailed information of siRNA pre-mix of IFITs
used for knocking-down experiments

| FlexiTube | Entrez | | | | |
|---|---|---|---|---|---|
| siRNA | Gene | | Target Sequence | Catalogue | |
| Premix | ID | Length | (5′ to 3′) | Number | SEQ ID NO: |
| Hs-IFIT1 | 3434 | 4396 bp | CAGGCTGTCCGCTTAAATCCA | S100445879 | SEQ ID NO: 7 |
| Hs-IFIT1 | 3434 | 4396 bp | TACATGGGAGTTATCCATTGA | S103224284 | SEQ ID NO: 8 |
| Hs-IFIT2 | 3433 | 3505 bp | AAAGAAAGTTACTGGAACTAA | S104145372 | SEQ ID NO: 9 |
| HS-IFIT2 | 3433 | 3505 bp | CCCATAGAGGTTAGTCCTGCA | S104259010 | SEQ ID NO: 10 |
| Hs-IFIT3 | 3437 | 2467 bp | ATGCTATGGACTATTCGAATA | S103152737 | SEQ ID NO: 11 |
| Hs-IFIT3 | 3437 | 2467 bp | AGAGATGATTGAAGCACTAAA | S104197788 | SEQ ID NO: 12 |

Determination of Bacterial Uptake after Knock-Up/Down of IFITs

Intracellular Colony Forming Units (CFUs) were determined in mycobacteria infected THP-1 cells knocked-up/down with IFITs family. These cells were lysed using 0.1% Triton X-100. Bacterial uptake was determined by serial dilution (10⁻¹-10⁻⁴) and plating out of mycobacteria onto 7H11 agar plates. The agar plates were incubated at 37° C. for 5 weeks and CFUs/ml was determined. Mycobacteria survival within the treated cells was monitored at 12 hours 96 hours post-infection respectively (with Mycobacterium smegmatis not measured at 96 hours).

Cytotoxicity Assay after Knock-Up/Down of IFITs

Cell cytotoxicity was tested with Roche Water Soluble Tetrazolium (WST-1) Cell Cytotoxicity Reagent (Roche, USA) in 1:10 dilution of WST-1 reagent to RPMI complete media (RPMI+10% Human Serum). Cells post-infection and respective time point incubation (12 and 96 hours) were processed for cell cytotoxicity. 300 μl of 1:10 dilution of WST-1 reagent to complete media was added to the wells of a 12-well plate. The culture plates were covered properly with aluminium foil as the cytotoxicity reagent is light sensitive. Cells were incubated for 1 hour at 37° C. and 5% CO₂. Cells were then transferred to a multi-mode reader placed in the dark room. Absorbance was measured at 450 and 630 nm (wavelength correction). The difference between the two absorbance readings was taken and plotted in Microsoft Excel as percentage values.

RNA Extraction after Knock-Up/Down of IFITs

Total RNA from human macrophages was extracted with the help of a kit RNeasy Plus Mini Kit (Cat. No. 74134, Qiagen, Limburg, Netherlands). The cell culture medium was completely aspirated from the culture plates. Cells were washed three times with ice-cold 1×PBS. 350 μl of RLT Plus Buffer (with 10 μl/ml β-mercaptoetanol) was added to the wells and scrapped with pipette tip to disrupt the cells. The lysate was then pipetted into a micro centrifuge tube and vortexed to ensure that no cell clumps are visible. The lysate was then loaded directly to a QIAshredder spin column and centrifuged for 2 minutes at maximum speed.

The homogenised lysate was then transferred to a gDNA Eliminator spin column and centrifuged for 1 minute at 8000×g. The gDNA Eliminator column ensures removal of any genomic DNA from all the samples. 350 μl of 70% ethanol was added to the flow through and mixed well by pipetting. Up to 700 μl of the sample (including any precipitate) was transferred to RNeasy spin column placed in a 2 ml collection tube and the lid was closed gently. This was then centrifuged for 15 seconds at 8000×g and the flow through was discarded.

700 μl of RW1 Buffer was then added to RNeasy spin column and the lid was gently closed. This was then centrifuged for 15 seconds at 8000×g to wash the spin column membrane and the flow through was discarded. 500 μl of RPE Buffer was then added to RNeasy spin column and the lid was gently closed. This was then centrifuged for 15 seconds at 8000×g to wash the spin column membrane and the flow through was discarded. 500 μl of RPE Buffer was then added to RNeasy spin column and the lid was gently closed. This was then centrifuged for 2 minutes at 8000×g to wash the spin column membrane and the flow through was discarded. The RNeasy spin column was then placed in a 2 ml collection tube and centrifuged at full speed for 1 minute. The RNeasy spin column was then placed in a 1.5 ml collection tube. 30 μl of RNase-free water was then added directly to the spin column membrane and then centrifuged for 1 minute at 8000×g to elute RNA.

For each experiment, RNA quantity and quality were measured using Agilent 2100 Bioanalyzer. The RNA with a high RNA integrity Number (RIN) 9) was used for Ampliseq and quantitative real time qPCR experiments.

Quantitative qPCR after Knock-Up/Down of IFITs

Good quality RNA (RIN>9, 0.8 μg) was used for cDNA preparation with the help of kit (Quantitect Reverse Transcription Kit). To ensure the removal of genomic DNA, ‘gDNA wipe-out buffer’ was added to RNA (included in the kit) prior to the RNA conversion step. qPCR amplification was run on a LightCycler 96 system (Roche, Germany). LightCycler 480 SYBR Green I Master was used for various differentially expressed genes using QuantiTect primer assays with 20 μl of reaction volume.

Hs-GAPDH and Hs-UBC were selected as reference genes confirmed to have stable expression levels. The primers used for amplification are shown in Table 3.

TABLE 3
Primer sequences used for confirmation of
stable expression levels.

| Reference | | | |
|---|---|---|---|
| Gene | Orientation | Sequence | SEQ ID NO: |
| Hs_GAPDH | Forward | TGCACCACCAAC | SEQ ID NO: 13 |
| | | TGCTTAGC | |
| Hs_GAPDH | Reverse | GGCATGGACTGT | SEQ ID NO: 14 |
| | | GGTCATGAG | |
| Hs_UBC | Forward | CGGTGAACGCCG | SEQ ID NO: 15 |
| | | ATGATTAT | |
| Hs_UBC | Reverse | ATCTGCATTGTC | SEQ ID NO: 16 |
| | | AAGTGACGA | |

The amplification process involved 45 cycles of 95° C. for 10 seconds followed by 60° C. for 10 seconds and finally 72° C. for 10 seconds. Gene expression fold-changes were computed for pathogenic infected and non-pathogenic infected macrophages using calibrated normalized relative quantities using the equation N=N₀×2^{cP}. All qPCRs were performed on RNA extracted from 3 additional experiments. All biological replicates having a positive control and a non-reverse transcription control was run in triplicate (along with calibrator) as per the MIQE Guidelines.

Western Blot

After the infection and subsequent transfection period, protein was extracted using RIPA buffer (0.5% sodium deoxycholate, 150 mM NaCl, 0.1% SDS and 50 mM Tris and 1.0% Triton X-100). 50 μl of protease inhibitor (Roche, Switzerland) was added to 1 ml of lysis buffer. Complete cell lysis was achieved by scraping the cells using a cell scraper and pipetting 5-10 times with a 1 ml pipet tip. Cells were left on ice for 10 minutes followed by centrifugation at 200×g for 10 minutes at 4° C., to achieve at least 80% cell lysis. The supernatant was collected and centrifuged at 8000×g for 30 minutes, the post-nuclear supernatant (PNS) was collected, filtered through a 0.22 μm syringe filter, PVDF (SIGMA-ALDRICH), USA) into 1.5 ml Eppendorf tubes and stored at −80° C. The protein quantity and quality was assessed by using the Bradford assay and SDS-PAGE.

Bradford assay: a standard curve was generated by using 10% Bovine serum albumin, Cat No. HD14-4 (BSA, QIAGEN, USA). A working stock of 1 mg/ml was made, 6 BSA concentrations used to generate the standard curve were 2 μg, 4 μg, 8 μg, 12 μg, 16 μg and 20 μg, 900 μl of 1× Bradford dye reagent Cat No. 500-0205 (Bio-Rad, USA) was added. Protein samples were kept on ice, 5 μl of the sample, 95 μl distilled water and 900 μl of 1× Bradford dye reagent were mixed to make 1 ml and absorbance taken at OD 595 on a spectrophotometer (MRCLAB Spectro UV-16, Israel). A control sample contained water and Bradford reagent.

10 μg of each protein sample was used. The sample was mixed with 1×XT sample buffer (Bio-Rad, USA) in an Eppendorf, heat at 90° C. on a heating block for 2 minutes. Loaded the sample in a 10% Precast SDS gel (Bio Rad Mini TGX Gels, Bio-Rad, USA, Cat No. 456-1044), in 1× running buffer XT MOPS, Bio-Rad, USA). The gel was run at 90 volts for quantification of the protein samples for 30 minutes. The gel was then transferred onto a PVDF membrane (BioRad, Cat No. 1620174) for 7 minutes using TURBO Blot Transfer machine (BioRad Trans Blot Turbo Transfer System, Serial No. 690BR016386). The blotted membranes were blocked with blocking solution (5% BSA in TBST buffer) for 2 hours at room temperature on a 15 rpm shaker. The membrane was then washed three times using TBST buffer (TBS—10×-24 g Tris base, 88 g NaCl, 900 ml of double distilled water, pH 7.6, mix and make up the volume to 1 litre with 0.1% Tween in TBST 1× Buffer). Each wash was performed for 5 minutes on a 20 rpm shaker at room temperature. Membrane was then incubated with respective primary antibody overnight at 4° C. (anti Flag antibody (Sigma Aldrich, Cat. No. F3165)—1:5000 dilution in TBST, anti IFIT1 antibody (Sigma Aldrich, Cat No. SAB4501508)—1:1000 dilution in TBST, anti IFIT2 antibody (Sigma Aldrich, Cat No. SAB2101128)—1:1000 dilution in TBST, anti IFIT3 antibody (Sigma Aldrich, Cat. No. AV46034)—1:1000 dilution in TBST). The membrane was washed three times using TBST buffer (each wash for 5 minutes on a 20 rpm shaker at room temperature. The membrane was then incubated with respective secondary antibody for 1 hour at room temperature (anti mouse monoclonal antibody (Santa Cruz Biotechnology, Sc516102)—1:5000 dilution in TBST used against anti FLAG Primary antibody and anti-rabbit monoclonal antibody (Santa Cruz Biotechnology, Sc2030)—1:5000 dilution in TBST used against anti IFIT1, anti IFIT2 and anti IFIT3 Primary antibody). The membrane was washed three times using TBST buffer (each wash for 5 minutes) on a 20 rpm shaker at room temperature. The membrane was conjugated with horseradish peroxidase (BioRad). The bound secondary antibody was spotted using an improved chemiluminescence detection kit Clarity Max Western ECL substrate (Cat. No. 1705062, BioRad). Furthermore, to ensure equal loading of proteins, the membranes were stripped for 30 minutes at room temperature with stripping buffer (100 mM 2-mercaptoethanol, 62.5 mM Tris, and pH 6.8, 2% SDS) and was re-probed with beta-actin/GAPDH antibody (Santa Cruz Biotechnology, Sc32233) for 2 hours at room temperature.

Statistical Analysis

Real time qPCR data were analysed using Light Cycler 96 SW 1.1 Software and Graph-pad Prism V7. Relative Expression which measures target transcript in a treatment group to that of the untreated group was measured through the software in response to the Calibrator and non-transcription control. The relative expression data of the cytokines was further analysed through Graph-pad prism to generate the p-values through One-Way ANOVA. The p-values were finally generated through Multiple Testing using Tukey corrections. The data (in technical triplicate) was finally plotted in histograms with respective mean and standard deviations.

Cytotoxicity graphs and CFUs were plotted with an average of the technical triplicates leading to the mean of all the Biological replicates. Statistical analysis was performed through Graph-pad Prism V7 software where the percentage of every expressing cell was generated and p-value was calculated using Two-Way ANOVA with Tukey's correction. Luminex data was analysed by Two-Way ANOVA with Tukey's correction using Graph-pad Prism V7 for Windows (Graph-pad Software, San Diego Calif., USA).

CFUs after Knocking-Up and Knocking-Down of IFITs

The inventors have determined CFUs of Mycobacterium smegmatis after 12 and 24 hours of infection, while CFUs of Mycobacterium bovis BCG and Mycobacterium tuberculosis R179 were determined after 12 and 96 hours of infection to human monocyte-derived macrophages (hMDMs) with knock-down and knock-up of IFIT1, IFIT2, and IFIT3. At 12 hours, the inventors found significantly higher (p<0.0001) CFUs after knock-down of all three IFITs, whereas, knock-up resulted in a significantly reduced (p<0.0001) number of CFUs for all three strains of mycobacteria.

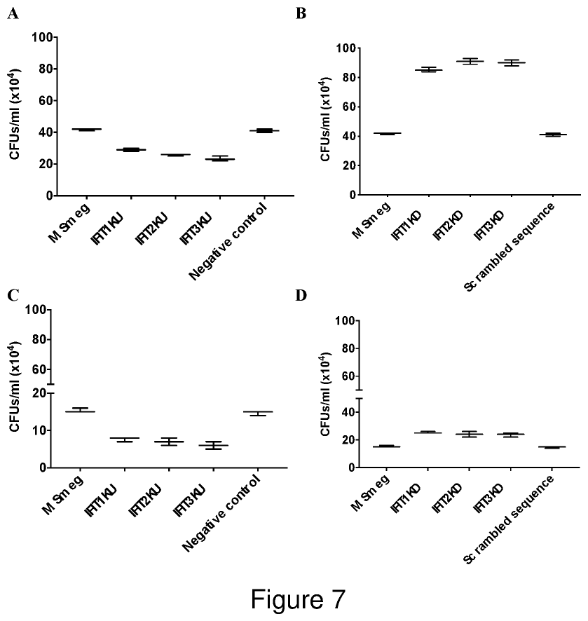

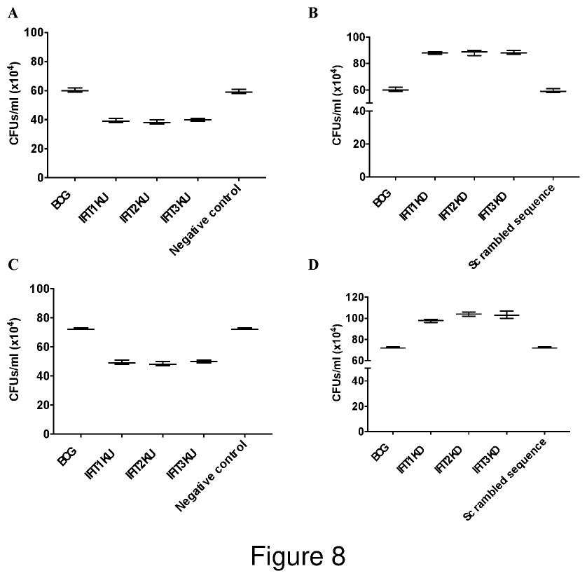

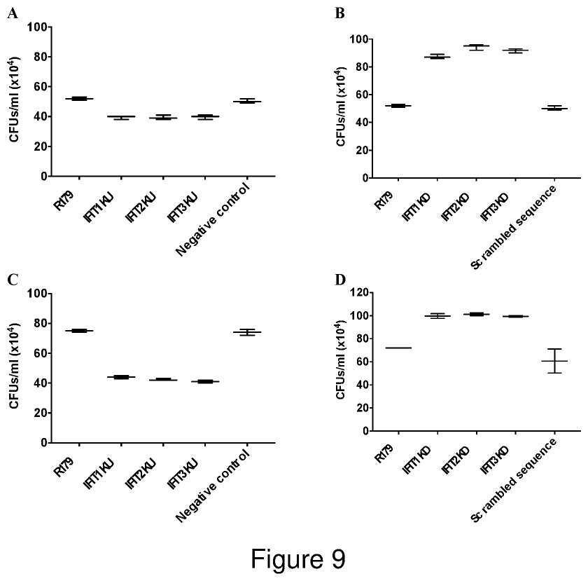

Subsequently, CFUs of Mycobacterium smegmatis at 24 hours, and Mycobacterium bovis BCG and Mycobacterium tuberculosis R179 at 96 hours post-infection upon knocking down with IFITs showed significantly higher CFUs (p<0.001), on the other hand, knocking-up, showed significantly reduced CFUs (p<0.001). Comparison of CFUs across the strains was found to be similar to the scrambled sequence for knock-down and negative control (vector) for knock-up of IFITs (FIGS. 7, 8 and 9).

At 12 hours, comparison of Mycobacterium smegmatis CFUs across knock down of IFIT family showed significantly higher counts after knock-down with IFIT2 (p=0.007) and IFIT3 (p=0.024) as compared to IFIT1.

At 96 hours (4 days), Mycobacterium bovis BCG CFUs across knock down of IFIT family showed significantly higher colony counts after knock-down with IFIT2 (p=0.018) and IFIT3 (p=0.034) as compared to IFIT1.

At 96 hours, comparison of Mycobacterium tuberculosis R179 CFUs across knock down of IFIT family showed higher colony counts after knock-down with IFIT2 (p=0.002) and IFIT3 (p=0.043) as compared to IFIT1.

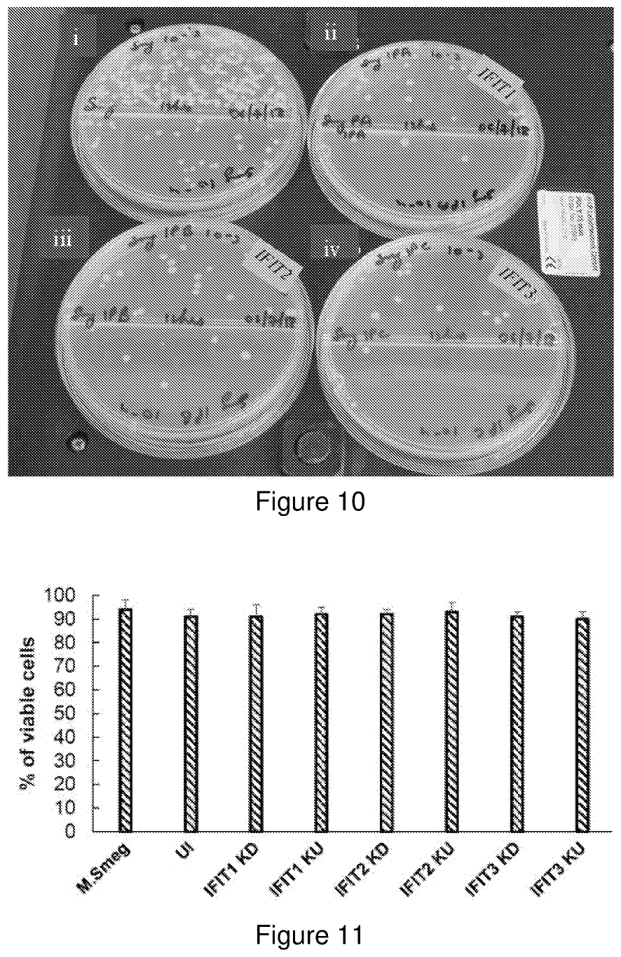

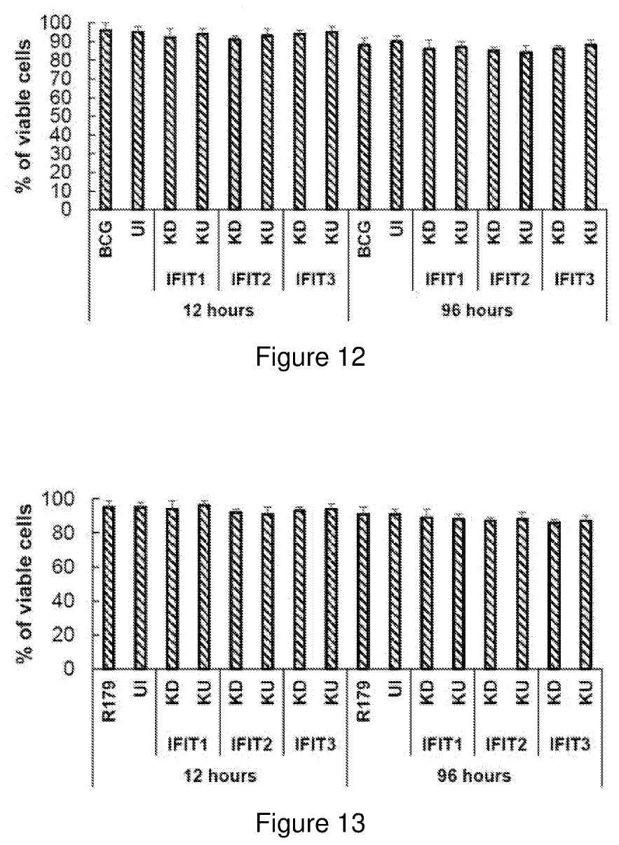

At 12 hours, comparison of CFUs across knock up of IFIT family showed significantly higher counts after knock-up with IFIT2 (p=0.014) and IFIT3 (p=0.001) as compared to IFIT1. FIG. 10 shows the representative image of CFUs after knocking-up of IFITs.

qPCR after Knock-Up/Down of IFITs

The inventors determined the relative expression of IFIT1, IFIT2 and IFIT3 upon infection with Mycobacterium smegmatis (at 12 hours), and Mycobacterium bovis BCG and Mycobacterium tuberculosis R179 (at 12 hours and 96 hours) through qPCR after knocking-down and knocking-up of these IFITs. This was carried out to standardize knocking-down and knocking-up of IFITs. Knocking-down IFITs across all three species showed significantly lower relative expression (p<0.001) and upon knocking-up IFITs showed statistically higher relative expression (p<0.001) of respective IFITs.

In Mycobacterium smegmatis, at 12 hours post-infection, the mRNA expression level for IFIT1 was very low upon knocking-down (p<0.001) while the expression level was higher upon knocking-up (p<0.001) when compared to the mRNA expression levels of cells only infected with Mycobacterium smegmatis. The mRNA expression level for IFIT2 was very low upon knocking-down (p<0.001) while the expression level was higher upon knocking-up (p<0.001) when compared to the mRNA expression levels of cells only infected with Mycobacterium smegmatis. This was also true for IFIT3 where the mRNA expression level was very low upon knocking-down (p<0.001) while the expression level was higher upon knocking-up (p<0.001) when compared to the mRNA expression levels of cells only infected with Mycobacterium smegmatis.

In Mycobacterium bovis BCG, at 12 hours post-infection, the mRNA expression level for IFIT1 was very low upon knocking-down (p<0.001) while, was higher upon knocking-up (p<0.001) when compared to the mRNA expression levels of cells only infected with Mycobacterium bovis BCG. The mRNA expression level for IFIT2 was very low upon knocking-down (p<0.001) while, was higher upon knocking-up (p<0.001) when compared to the mRNA expression levels of cells only infected with Mycobacterium bovis BCG. This was also true for IFIT3 where the mRNA expression level was very low upon knocking-down (p<0.001) while, was higher upon knocking-up (p<0.001) when compared to the mRNA expression levels of cells only infected with Mycobacterium bovis BCG. Upon knocking-up, mRNA expression level was higher for IFIT2 (p<0.036) as compared to IFIT1 and IFIT3.

In Mycobacterium bovis BCG, at 96 hours post-infection, the mRNA expression level for IFIT1 was very low upon knocking-down (p<0.001) while, was higher upon knocking-up (p<0.001) when compared to the mRNA expression levels of cells only infected with Mycobacterium bovis BCG. The mRNA expression level for IFIT2 was very low upon knocking-down (p<0.001) while, was higher upon knocking-up (p<0.001) when compared to the mRNA expression levels of cells only infected with Mycobacterium bovis BCG. This was also true for IFIT3 where the mRNA expression level was very low upon knocking-down (p<0.001) while, was higher upon knocking-up (p<0.001) when compared to the mRNA expression levels of cells only infected with Mycobacterium bovis BCG.

In Mycobacterium tuberculosis R179, at 12 hours post-infection, the mRNA expression level for IFIT1 was very low upon knocking-down (p<0.001) while, was higher upon knocking-up (p<0.001) when compared to the mRNA expression levels of cells only infected with Mycobacterium tuberculosis R179. The mRNA expression level for IFIT2 was very low upon knocking-down (p<0.001) while, was higher upon knocking-up (p<0.001) when compared to the mRNA expression levels of cells only infected with Mycobacterium tuberculosis R179. This was also true for IFIT3 where the mRNA expression level was very low upon knocking-down (p<0.001) while, was higher upon knocking-up (p<0.001) when compared to the mRNA expression levels of cells only infected with Mycobacterium tuberculosis R179.

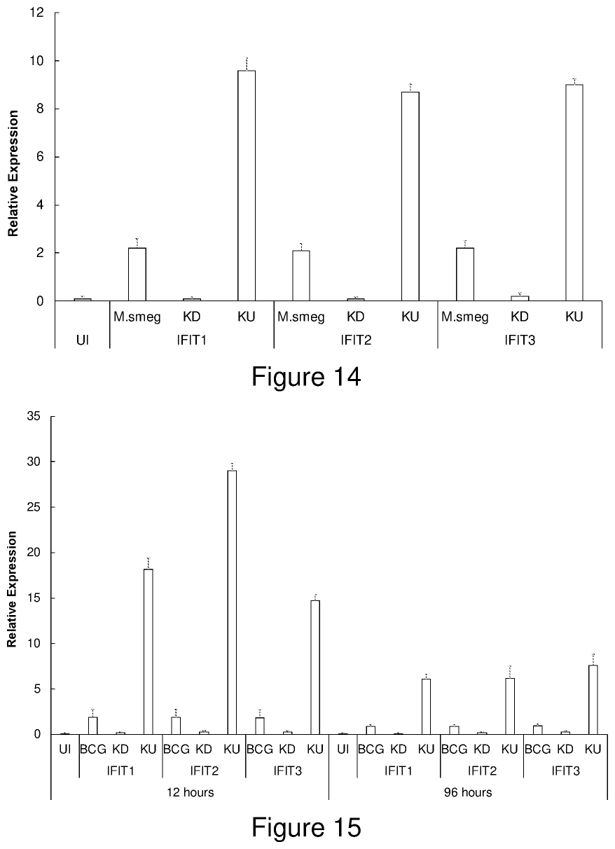

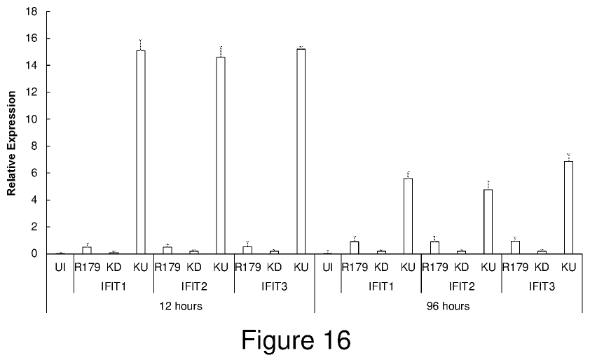

In Mycobacterium tuberculosis R179, at 96 hours post-infection, the mRNA expression level for IFIT1 was very low upon knocking-down (p<0.001) while, was higher upon knocking-up (p<0.001) when compared to the mRNA expression levels of cells only infected with Mycobacterium tuberculosis R179. The mRNA expression level for IFIT2 was very low upon knocking-down (p<0.001) while, was higher upon knocking-up (p<0.001) when compared to the mRNA expression levels of cells only infected with Mycobacterium tuberculosis R179. This was also true for IFIT3 where the mRNA expression level was very low upon knocking-down (p<0.001) while, was higher upon knocking-up (p<0.001) when compared to the mRNA expression levels of cells only infected with Mycobacterium tuberculosis R179. FIGS. 14, 15 and 16 show the comparison of mRNA expression upon knocking-up and knocking-down of IFITs in hMDMs after infection with Mycobacterium smegmatis, Mycobacterium bovis BCG and Mycobacterium tuberculosis R179 respectively.

Western Blot after Knock-Up/Down of IFITs

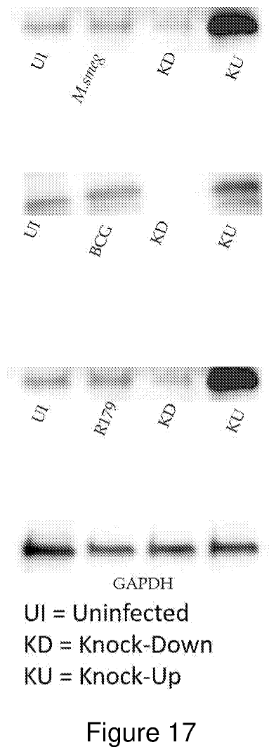

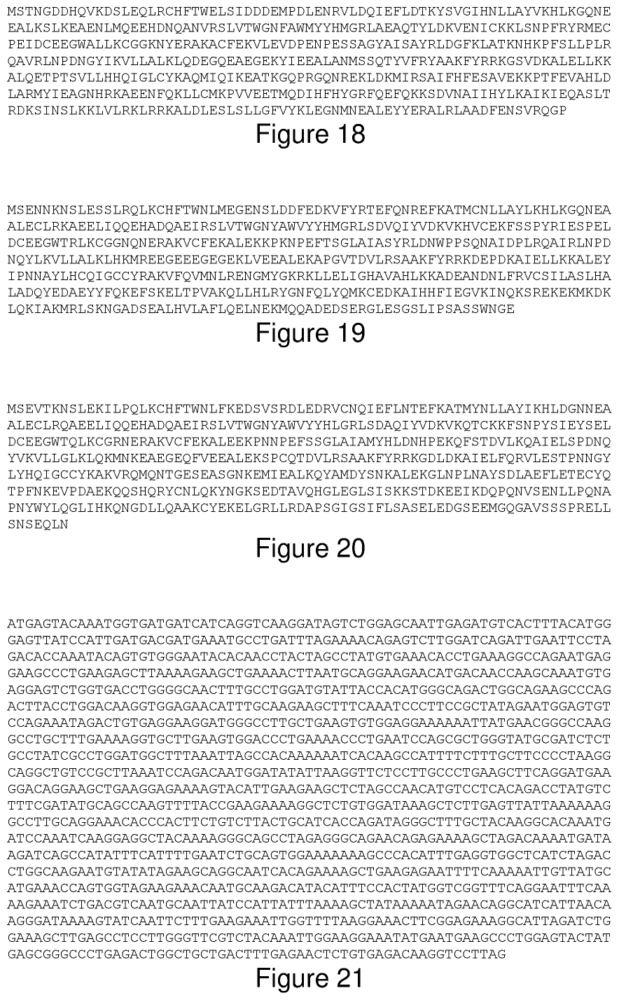

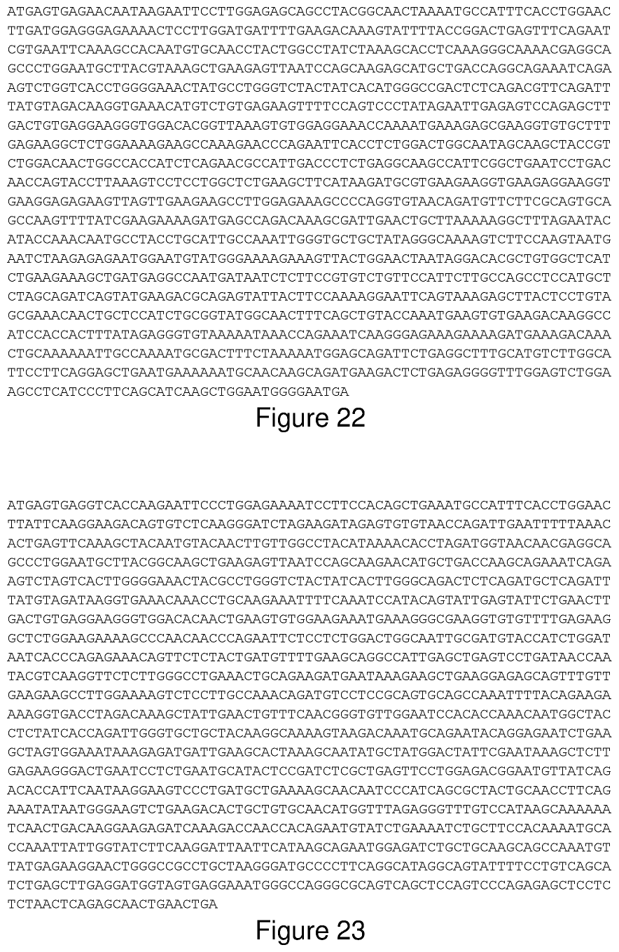

The inventors also confirmed knocking-up and knocking-down of IFITs by investigating protein expression through Western blotting (FIG. 17). GAPDH protein expression was used as an internal quality control. The blots clearly depict that after knocking-up there was an increase in band intensity across all three species. Whereas, the inventors observed a decrease in intensity after knocking down across the species of mycobacteria.

### Example 2

The inventors conducted a detailed investigation of in-vitro host response from human monocyte derived macrophages (hMDMs) towards different strains of mycobacteria (grown in detergent-free media), i.e. pathogenic (Mycobacterium tuberculosis R179) and non-pathogenic species (Mycobacterium smegmatis and Mycobacterium bovis BCG). The host response was measured post-infection (at mRNA and protein levels) using amplicons-based RNA sequencing (AmpliSeq), quantitative real time polymerase chain reaction (RTqPCR), multiplex ELISA (Luminex), intracellular mycobacterial survival and cytotoxicity assays.

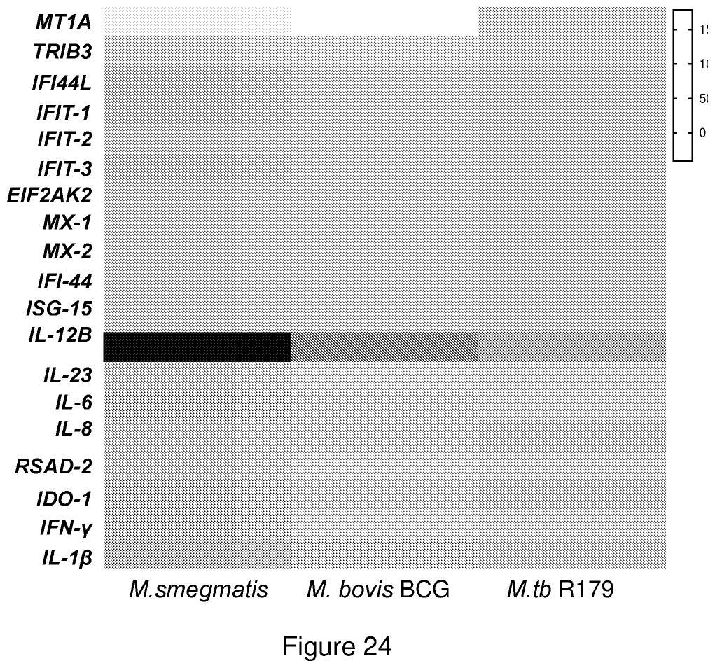

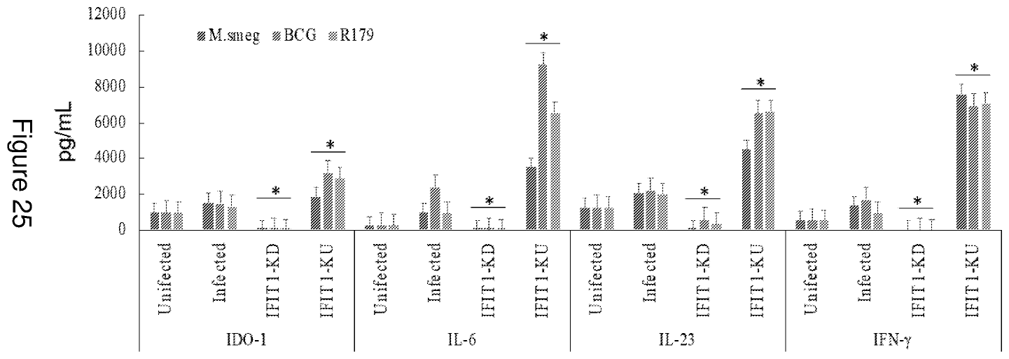

Biological network analysis (ingenuity pathway analysis IPA) was performed to understand the gene regulatory network involved in the pathophysiology associated with the host-immune system. Based on false discovery rate (FDR) and biological functions, the inventors selected 19 potential differentially expressed genes (DEGs) (FIG. 24). Out of these, an inter-related gene family of interferon induced protein with tetratricopeptides (IFIT1, IFIT2 and IFIT3) was used to study mycobacterial intervention experiments in THP-1 cells.

The IFIT gene family is known to form a protein complex during viral infection to act against the antigen. Study encompassing the role of IFITs against mycobacteria is not well established. Therefore, the inventors performed in-vitro vector-based overexpression (knock-up) and small interfering RNA (siRNA) approach (knock-down) of IFITs to investigate their effect upon mycobacteria inside the host macrophages.

AmpliSeq analysis found 19 differentially expressed genes at 12 hours post-infection across all three mycobacterial species (FIG. 24). Lower numbers of intra-cellular mycobacterial growth (CFUs) and higher host response in hMDMs infected with Mycobacterium smegmatis were observed as compared to other two mycobacterial species. In the biological network analysis, we found interferon-interleukin associated signalling pathways as most prominent among the 19 differentially expressed genes.

A differed host response towards all three species was observed which may attribute to their pathogenicity. mRNA and protein level comparisons at different time points, depicted strong role of interferon and interleukin associated gene network. This network was able to successfully counter Mycobacterium smegmatis but succumb to Mycobacterium bovis BCG and Mycobacterium tuberculosis R179. The intervention experiments were carried out in THP-1 like macrophages. A pictorial representation of in-vitro knock-up using vector-based transfection is depicted in FIG. 10. A Western blot was performed to confirm protein expression upon knock-up/down of IFITs where GAPDH was used as an internal control (FIG. 17).

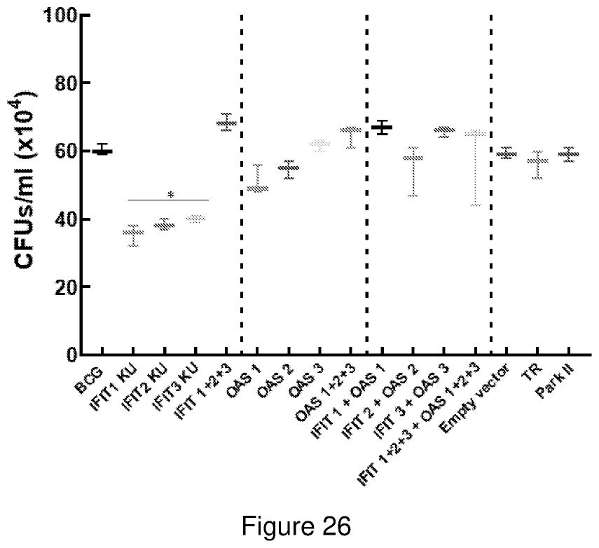

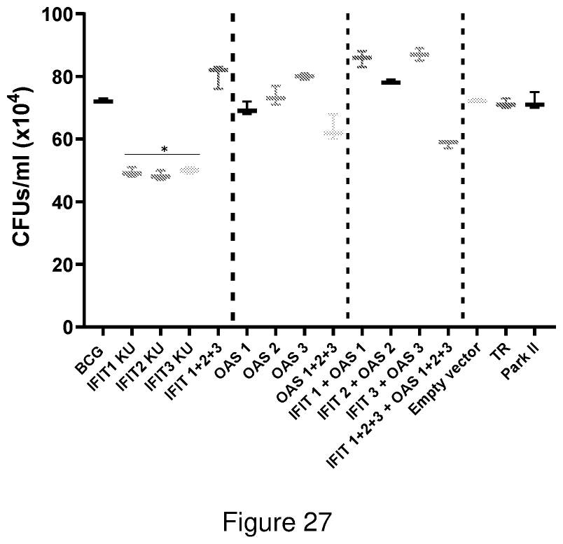

Synergy experiments of IFITs and OASs were performed to study their effect upon mycobacteria (Leisching et. al. 2019). Surprisingly, the combinational therapy of IFIT1, IFIT2 and IFIT3 together did not show any effect upon knock-up/down as depicted by individual IFITs (FIGS. 26 and 27). Previous study in our group observed OASs effect upon mycobacteria. It is noteworthy that individual IFIT Ttreatment is more promising than any other gene or their combination.

The inventors found higher expression of key pro-inflammatory cytokines (i.e. IDO1, IFN-γ, IL-6, and IL-23) during knock-up resulting in reduction of mycobacteria (FIG. 25). It is well established that Mycobacterium tuberculosis interferes with host signalling pathways activated with IFN-γ for its survival inside macrophages.

It is noteworthy, the levels of IFN-γ increased further (even significantly higher than usual infection) after vector-based knock-up (vector-based over-expression) of IFITs. On the other hand, IFITs knock-down completely eliminated IFN-γ expression. Similar to IFN-γ, higher levels of IL-6 were also found after IFITs knock-up (vector-based over-expression). IL-6 has been shown to be differentially expressed as part of a protective immune response in Mycobacterium tuberculosis infected mice.

But similar to IDO-1, IL-6 is not essential for anti-mycobacterial mechanisms. IL-23 is found to induce IL-17 levels from T helper cells in healthy tuberculin reactors. Increased IL-23 levels in our study indicates reducing intracellular survival of Mycobacterium tuberculosis (reduced CFUs). This can be attributed to induction of IL-17 and IFN-γ cytokines through IL-23.

Our results show that IFITs at individual level play an important role in mycobacterial killing. Knock-up of individual IFITs inside macrophages causes a significant increase in key pro-inflammatory cytokines (IDO-1, IL-6, IL-23 and IFN-γ) resulting in mycobacterial killing. Knock-down of IFITs inside macrophages causes a significant decrease in key pro-inflammatory cytokines (IDO-1, IL-6, 11-23 and IFN-γ) resulting in mycobacterial survival.

Differentially expressed IFITs showed a strong effect against mycobacteria, which can be used as a promising therapeutic target adjunct to anti-TB therapy. This knowledge will broaden the scope of host drug targets for resistance free bacteriostatic immuno-therapy.

### REFERENCES

Der S D, Zhou A, Williams B R G, Silverman R H. Identification of genes differentially regulated by interferon α, β, or γ using oligonucleotide arrays. Proc Natl Acad Sci USA. 1998 Dec 22; 95(26):15623-8.
Fensterl V, Sen G C. The ISG56/IFIT1 gene family. J Interferon Cytokine Res Off J Int Soc Interferon Cytokine Res. 2011 January; 31(1):71-8.
Harper C J, Hayward D, Kidd M, Wiid I, van Helden P. Glutamate dehydrogenase and glutamine synthetase are regulated in response to nitrogen availability in Myocbacterium smegmatis. BMC Microbiol. 2010 May 11; 10(1):138.
Johnson R, Warren R M, van der Spuy G D, Gey van Pittius N C, Theron D, Streicher E M, et al. Drug-resistant tuberculosis epidemic in the Western Cape driven by a virulent Beijing genotype strain. Int J Tuberc Lung Dis Off J Int Union Tuberc Lung Dis. 2010 January; 14(1):119-21.
Katibah G E, Lee H J, Huizar J P, Vogan J M, Alber T, Collins K. tRNA binding, structure, and localization of the human interferon-induced protein IFIT5. Mol. Cell. 2013 Feb. 21; 49(4):743-50.
Kumar P, Sweeney T R, Skabkin M A, Skabkina O V, Hellen C U T, Pestova T V. Inhibition of translation by IFIT family members is determined by their ability to interact selectively with the 5′-terminal regions of cap0-, cap1- and 5′ppp-mRNAs. Nucleic Acids Res. 2014 March; 42(5):3228-45.
Kusari J, Sen G C. Regulation of synthesis and turnover of an interferon-inducible mRNA. Mol Cell Biol. 1986 June; 6(6):2062-7.
Leisching G, Cole V, Ali A T, Baker B. OAS1, OAS2 and OAS3 restrict intracellular M. tb replication and enhance cytokine secretion. Int J Infect Dis. 2019 March; 80S:S77-S84. doi: 10.1016/j.ijid.2019.02.029.
Viljoen A J, Kirsten C J, Baker B, van Helden P D, Wiid I J F. The role of glutamine oxoglutarate aminotransferase and glutamate dehydrogenase in nitrogen metabolism in Mycobacterium bovis BCG. PloS One. 2013; 8(12):e84452.

1. A method of increasing the cellular concentration of an Interferon Induced Protein with Tetratricopeptide repeats (IFIT) polypeptide in a cell infected with a mycobacterium, the method comprising introducing an exogenous IFIT polypeptide or an expression vector comprising a polynucleotide encoding the exogenous IFIT polypeptide into the cell, wherein the exogenous IFIT polypeptide is selected from at least one exogenous IFIT1, IFIT2 or IFIT3 polypeptide, and wherein increasing the cellular concentration of the IFIT polypeptide reduces the number of viable mycobacteria in the cell.

2. The method of claim 1, wherein the exogenous IFIT polypeptide comprises an amino acid sequence of SEQ ID NO:1, SEQ ID NO:2 or SEQ ID NO:3.

3. The method of claim 1, wherein the polynucleotide encoding the exogenous IFIT polypeptide comprises a nucleic acid sequence of SEQ ID NO:4, SEQ ID NO:5 or SEQ ID NO:6.

4. The method of claim 1, further comprising introducing a pharmaceutical compound selected from the group consisting of arginine, amikacin, bedaquiline, capreomycin, ciprofloxacin, clarithromycin, clavulanic acid, clofazimine, co-amoxiclav, cycloserine, enviomycin, ethambutol, ethionamide, imipenem, interferon-γ, isoniazid, kanamycin, levofloxacin, linezolid, meropenem, metronidazole, moxifloxacin, para-aminosalicylic acid, prochlorperazine, prothionamide, pyrazinamide, rifabutin, rifampicin, streptomycin, thioacetazone, thioridazine, vitamin D, and viomycin to the cell.

5. The method of claim 1, wherein the mycobacterium is selected from the group consisting of Mycobacterium smegmatis, Mycobacterium bovis, and Mycobacterium tuberculosis.

6. The method of claim 1, wherein the cell is an immune cell, such as a macrophage.

7. A method of treating a mycobacterial infection in a subject, the method comprising administering a therapeutically effective amount of:
(i) an exogenous Interferon Induced Protein with Tetratricopeptide repeats (IFIT) polypeptide, or
(ii) an expression vector comprising a polynucleotide encoding the exogenous IFIT polypeptide
to the subject, thereby increasing the cellular concentration of the IFIT polypeptide in a cell infected with a mycobacterium;
wherein the exogenous IFIT polypeptide is selected from at least one exogenous IFIT1, IFIT2 or IFIT3 polypeptide, and
wherein increasing the cellular concentration of the IFIT polypeptide reduces the number of viable mycobacteria in the cell, thereby treating the mycobacterial infection.

8. The method of claim 7, wherein the exogenous IFIT polypeptide comprises an amino acid sequence of SEQ ID NO:1, SEQ ID NO:2 or SEQ ID NO:3.

9. The method of claim 7, wherein the polynucleotide encoding the exogenous IFIT polypeptide comprises a nucleic acid sequence of SEQ ID NO:4, SEQ ID NO:5 or SEQ ID NO:6.

10. The method of claim 7, further comprising administering a pharmaceutical compound selected from the group consisting of arginine, amikacin, bedaquiline, capreomycin, ciprofloxacin, clarithromycin, clavulanic acid, clofazimine, co-amoxiclav, cycloserine, enviomycin, ethambutol, ethionamide, imipenem, interferon-γ, isoniazid, kanamycin, levofloxacin, linezolid, meropenem, metronidazole, moxifloxacin, para-aminosalicylic acid, prochlorperazine, prothionamide, pyrazinamide, rifabutin, rifampicin, streptomycin, thioacetazone, thioridazine, vitamin D, and viomycin to the subject.

11. The method of claim 10, wherein the pharmaceutical compound and the IFIT polypeptide are administered to the subject separately, concurrently or sequentially.

12. The method of claim 7, wherein the mycobacterium is selected from the group consisting of Mycobacterium smegmatis, Mycobacterium bovis, and Mycobacterium tuberculosis.

13. The method of claim 7, wherein the cell is an immune cell, such as a macrophage.

14. The method of claim 7, wherein the subject is selected from the group consisting of a reptile, bird or mammal.

15. The method of claim 14, wherein the subject is a human.

16.-24. (canceled)


SEQUENCE LISTING (16 sequences)

SEQ ID NO: 1 - protein, 478 aa, Homo sapiens
MSTNGDDHQVKDSLEQLRCHFTWELSIDDDEMPDLENRVLDQIEFLDTKYSVGIHNLLAYVKHLKGQNEEALKSLKEAENLMQEEHDNQANVRSLVTWGNFAWMYYHMGRLAEAQTYLDKVENICKKLSNPFRYRMECPEIDCEEGWALLKCGGKNYERAKACFEKVLEVDPENPESSAGYAISAYRLDGFKLATKNHKPFSLLPLRQAVRLNPDNGYIKVLLALKLQDEGQEAEGEKYIEEALANMSSQTYVFRYAAKFYRRKGSVDKALELLKKALQETPTSVLLHHQIGLCYKAQMIQIKEATKGQPRGQNREKLDKMIRSAIFHFESAVEKKPTFEVAHLDLARMYIEAGNHRKAEENFQKLLCMKPVVEETMQDIHFHYGRFQEFQKKSDVNAIIHYLKAIKIEQASLTRDKSINSLKKLVLRKLRRKALDLESLSLLGFVYKLEGNMNEALEYYERALRLAADFENSVRQGP

SEQ ID NO: 2 - protein, 472 aa, Homo sapiens
MSENNKNSLESSLRQLKCHFTWNLMEGENSLDDFEDKVFYRTEFQNREFKATMCNLLAYLKHLKGQNEAALECLRKAEELIQQEHADQAEIRSLVTWGNYAWVYYHMGRLSDVQIYVDKVKHVCEKFSSPYRIESPELDCEEGWTRLKCGGNQNERAKVCFEKALEKKPKNPEFTSGLAIASYRLDNWPPSQNAIDPLRQAIRLNPDNQYLKVLLALKLHKMREEGEEEGEGEKLVEEALEKAPGVTDVLRSAAKFYRRKDEPDKAIELLKKALEYIPNNAYLHCQIGCCYRAKVFQVMNLRENGMYGKRKLLELIGHAVAHLKKADEANDNLFRVCSILASLHALADQYEDAEYYFQKEFSKELTPVAKQLLHLRYGNFQLYQMKCEDKAIHHFIEGVKINQKSREKEKMKDKLQKIAKMRLSKNGADSEALHVLAFLQELNEKMQQADEDSERGLESGSLIPSASSWNGE

SEQ ID NO: 3 - protein, 490 aa, Homo sapiens
MSEVTKNSLEKILPQLKCHFTWNLFKEDSVSRDLEDRVCNQIEFLNTEFKATMYNLLAYIKHLDGNNEAALECLRQAEELIQQEHADQAEIRSLVTWGNYAWVYYHLGRLSDAQIYVDKVKQTCKKFSNPYSIEYSELDCEEGWTQLKCGRNERAKVCFEKALEEKPNNPEFSSGLAIAMYHLDNHPEKQFSTDVLKQAIELSPDNQYVKVLLGLKLQKMNKEAEGEQFVEEALEKSPCQTDVLRSAAKFYRRKGDLDKAIELFQRVLESTPNNGYLYHQIGCCYKAKVRQMQNTGESEASGNKEMIEALKQYAMDYSNKALEKGLNPLNAYSDLAEFLETECYQTPFNKEVPDAEKQQSHQRYCNLQKYNGKSEDTAVQHGLEGLSISKKSTDKEEIKDQPQNVSENLLPQNAPNYWYLQGLIHKQNGDLLQAAKCYEKELGRLLRDAPSGIGSIFLSASELEDGSEEMGQGAVSSSPRELLSNSEQLN

SEQ ID NO: 4 - DNA, 1437 bp, Homo sapiens
atgagtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaaaaattatgaacgggccaaggcctgctttgaaaaggtgcttgaagtggaccctgaaaaccctgaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttccactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctacaaattggaaggaaatatgaatgaagccctggagtactatgagcgggccctgagactggctgctgactttgagaactctgtgagacaaggtccttag

SEQ ID NO: 5 - DNA, 1419 bp, Homo sapiens
atgagtgagaacaataagaattccttggagagcagcctacggcaactaaaatgccatttcacctggaacttgatggagggagaaaactccttggatgattttgaagacaaagtattttaccggactgagtttcagaatcgtgaattcaaagccacaatgtgcaacctactggcctatctaaagcacctcaaagggcaaaacgaggcagccctggaatgcttacgtaaagctgaagagttaatccagcaagagcatgctgaccaggcagaaatcagaagtctggtcacctggggaaactatgcctgggtctactatcacatgggccgactctcagacgttcagatttatgtagacaaggtgaaacatgtctgtgagaagttttccagtccctatagaattgagagtccagagcttgactgtgaggaagggtggacacggttaaagtgtggaggaaaccaaaatgaaagagcgaaggtgtgctttgagaaggctctggaaaagaagccaaagaacccagaattcacctctggactggcaatagcaagctaccgtctggacaactggccaccatctcagaacgccattgaccctctgaggcaagccattcggctgaatcctgacaaccagtaccttaaagtcctcctggctctgaagcttcataagatgcgtgaagaaggtgaagaggaaggtgaaggagagaagttagttgaagaagccttggagaaagccccaggtgtaacagatgttcttcgcagtgcagccaagttttatcgaagaaaagatgagccagacaaagcgattgaactgcttaaaaaggctttagaatacataccaaacaatgcctacctgcattgccaaattgggtgctgctatagggcaaaagtcttccaagtaatgaatctaagagagaatggaatgtatgggaaaagaaagttactggaactaataggacacgctgtggctcatctgaagaaagctgatgaggccaatgataatctcttccgtgtctgttccattcttgccagcctccatgctctagcagatcagtatgaagacgcagagtattacttccaaaaggaattcagtaaagagcttactcctgtagcgaaacaactgctccatctgcggtatggcaactttcagctgtaccaaatgaagtgtgaagacaaggccatccaccactttatagagggtgtaaaaataaaccagaaatcaagggagaaagaaaagatgaaagacaaactgcaaaaaattgccaaaatgcgactttctaaaaatggagcagattctgaggctttgcatgtcttggcattccttcaggagctgaatgaaaaaatgcaacaagcagatgaagactctgagaggggtttggagtctggaagcctcatcccttcagcatcaagctggaatggggaatga

SEQ ID NO: 6 - DNA, 1473 bp, Homo sapiens
atgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactga

SEQ ID NO: 7 - DNA, 21 bp, Artificial Sequence
  Nucleic acid target sequence 1 of IFIT1 (Catalogue Number S100445879)
caggctgtccgcttaaatcca

SEQ ID NO: 8 - DNA, 21 bp, Artificial Sequence
  Nucleic acid target sequence 2 of IFIT1 (Catalogue Number S103224284)
tacatgggagttatccattga

SEQ ID NO: 9 - DNA, 21 bp, Artificial Sequence
  Nucleic acid target sequence 1 of IFIT2 (Catalogue Number S104145372)
aaagaaagttactggaactaa

SEQ ID NO: 10 - DNA, 21 bp, Artificial Sequence
  Nucleic acid target sequence 2 of IFIT2 (Catalogue Number S104259010)
cccatagaggttagtcctgca

SEQ ID NO: 11 - DNA, 21 bp, Artificial Sequence
  Nucleic acid target sequence 1 of IFIT3 (Catalogue Number S103152737)
atgctatggactattcgaata

SEQ ID NO: 12 - DNA, 21 bp, Artificial Sequence
  Nucleic acid target sequence 2 of IFIT3 (Catalogue Number S104197788)
agagatgattgaagcactaaa

SEQ ID NO: 13 - DNA, 20 bp, Artificial Sequence
  Nucleic acid sequence of Hs_GAPDH Forward primer
tgcaccaccaactgcttagc

SEQ ID NO: 14 - DNA, 21 bp, Artificial Sequence
  Nucleic acid sequence of Hs_GAPDH Reverse primer
ggcatggactgtggtcatgag

SEQ ID NO: 15 - DNA, 20 bp, Artificial Sequence
  Nucleic acid sequence of Hs_UBC Forward primer
cggtgaacgccgatgattat

SEQ ID NO: 16 - DNA, 21 bp, Artificial Sequence
  Nucleic acid sequence of Hs_UBC Reverse primer
atctgcattgtcaagtgacga